(19) United States
(12) Patent Application Publication
(10) Pub. No.: US 2025/0235453 A1
(43) Pub. Date: Jul. 24, 2025

(54) TRIAZOLOPYRIDAZINE COMPOUNDS USEFUL AS RAC1 INHIBITORS

(71) Applicant: Revere Pharmaceuticals, Arlington, MA (US)

(72) Inventors: Erik T. Goka, Arlington, MA (US); I. Robert Silverman, Arlington, MA (US); Alan Bruce Cooper, West Caldwell, NJ (US)

(21) Appl. No.: 18/704,418

(22) PCT Filed: Oct. 25, 2022
(86) PCT No.: PCT/US2022/047717

§ 371 Date: Apr. 24, 2024

Related U.S. Application Data
(60) Provisional application No. 63/271,478, filed on Oct. 25, 2021.
(60) Provisional application No. 63/301,246, filed on Jan. 20, 2022.

(51) Int. Cl. A61K 31/5025

(52) U.S. Cl. CPC A61K 31/5025

(57) ABSTRACT

Disclosed is a pharmaceutical composition comprising a pharmaceutically acceptable excipient, carrier or diluent and a compound of Formula I: or a pharmaceutically acceptable salt thereof. The variables are defined herein. Also disclosed is a method of treating cancer with the pharmaceutical formulation.

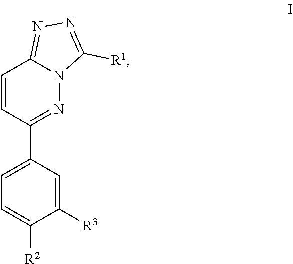

### CROSS-REFERENCE TO RELATED APPLICATIONS

This application claims the benefit of priority to U.S. Provisional Application No. 63/271,478, filed on Oct. 25, 2021, and U.S. Provisional Application No. 63/301,246, filed on Jan. 20, 2022. The entire contents of each of the aforementioned applications are incorporated herein by reference.

### BACKGROUND

The Rho GTPases Rae (Ras-related C3 botulinum toxin substrate) and Cdc42 (cell division control protein 42 homolog) regulate many cell functions, including cell polarity, migration, and cell cycle progression. The Rho family of GTPases in humans consists of 20 different members, and aberrant behavior in their regulatory activity has been implicated in cancer and other diseases. More than 70 guanine nucleotide exchange factors (GEFs) are known, which specifically activate one or more of the GTPases. In turn, the activated GTPases can specifically interact with over 60 downstream effectors. Dysregulation of one or more cellular processes can lead to release of malignant cells from their original locations, which subsequently can establish themselves in pre-metastatic niches in, for example, bone or lungs. It has been found that members of the Rho GTPase family, including Rae, Cdc42 and Rho, play key signaling roles in these processes.

Rho GTPases regulate migration and invasion, cytoskeletal organization, transcriptional regulation, cell cycle progression, apoptosis, vesicle trafficking, and cell-to-cell and cell-to-extracellular matrix adhesions. The Rho GTPases Rac and Cdc42 are potent inducers of actin polymerization and extension of actin structures at the leading edge of motile cells. In addition, Cdc42 plays a critical role in cell polarity, and thus, promotes directed and persistent migration.

Hyperactive Rae and Cdc42 are associated with increased cancer cell survival, proliferation, and invasion, as well as Ras and other oncogene-mediated transformation. Furthermore, oncogenic cell surface receptors, such as tyrosine kinase, cytokine, and G protein coupled receptors, activate Rae and Cdc42 via regulation of their upstream effector GEFs.

Despite the recognized role of Rac1 in promoting tumor progression, there are no approved drugs that target this signaling protein. Although a handful of Rac1 inhibitors have been reported, the reported inhibitors have not been suitable for clinical development due to low potency or poor drug properties.

NSC23766 was identified as a small molecule that binds to a putative binding pocket in the surface groove of Rac1 that interacts with the Rac-specific GEFs Trio and Tiam1.

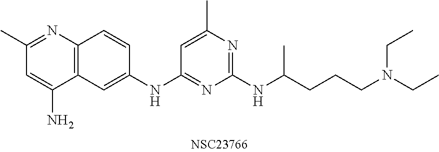 [SMILES: CCN(CC)CCCC(C)Nc1nc(C)cc(Nc2ccc3nc(C)cc(N)c3c2)n1]

NSC23766 has been shown to inhibit the anchorage-independent growth and invasion of human prostate cancer PC-3 cells as well as Rae activation and Rac-dependent aggregation of platelets stimulated by thrombin. It also inhibits Rac1 and Rac2 activities of hematopoietic stem/progenitor cells and migration from mouse bone marrow to peripheral blood. NSC23766 has also been shown to inhibit invasion of chronic myelogenous leukemia cells in vitro and in vivo in a mouse model. However, NSC23766 is a relatively weak Rae inhibitor, with a high IC₅₀ of 50-100 μM in fibroblasts. The weak activity of NSC23766 limits its potential use as a therapeutic agent.

U.S. Pat. No. 8,884,006 discloses a derivative of NSC23766, EHop-016, that is a more potent inhibitor of Rac1

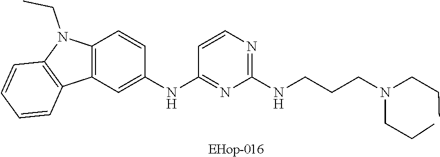 [SMILES: CCCN(CCC)CCCNc1nccc(Nc2ccc3c(c2)c2ccccc2n3CC)n1]

EHop-016 is reported to be 100-fold more efficient than NSC23766 as an inhibitor of Rae activity. In MDA-MB-435 breast cancer cells, EHop-016 (<5 mM) inhibits the association of the Rac-GEF Vav2 with a nucleotide-free Rac I (G15A), which has a high affinity for activated GEFs. EHop-016 does not affect the association of the Rac-GEF Tiam-1 with Rac1 (G15A) at similar concentrations. EHop-016 also inhibits the Rac activity of MDA-MB-231 metastatic breast cancer cells and reduces Rac-directed lamellipodia formation in both cell lines. Despite its improved potency, EHop-016 does not have a favorable in vivo pharmacokinetic profile, with low systemic exposure after oral administration in mice. Humphries-Bickley et al. J Chromatography 13 (2015), Volume 981-982, 19-26.

U.S. Pat. No. 1,047,235 and Molecular Cancer Therapeutics (2019). 18(5), 957-968 describes GYS32661:

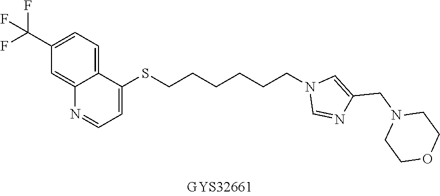 [SMILES: FC(F)(F)c1ccc2c(SCCCCCCn3cnc(CN4CCOCC4)c3)ccnc2c1]

GYS32661 shows very good activity against animal models of estrogen positive and HER2 positive breast cancer, prostate cancer, melanoma and colorectal cancer as a single agent and in combination with standard of care. GYS32661 has a relatively short plasma half-life and is not orally bioavailable.

While Rae and Cdc42 GTPases are hyperactive or overexpressed in many types of cancer, there are no drugs for these important targets. As there is a continuing need for new therapeutic agents to treat cancer and other hyperproliferative diseases, it is desirable to have new inhibitors of Rae and/or Cdc42 with improved activity and pharmacokinetic properties.

### SUMMARY OF THE INVENTION

It has now been found that certain triazolopyridazine compounds disclosed herein have potent activity against wild type Rac1 and certain genomic variants thereof such as Rac1b and Rac1 P29S. A number of these compounds are significantly more potent than the aforementioned prior art compounds with an IC₅₀ inhibitory concentration versus wild type Rac1 below 1.0 μM in an AlphaLisa assay (see the section entitled “In Vitro Assays” below subsection 2 and Tables 1-6). By comparison, the reference standard Ehop-016 has an IC₅₀ of about 10 μM in the same assay. Based on this discovery, pharmaceutical compositions comprising the disclosed Rac1 inhibitors and methods of treating cancer with the disclosed Rac1 inhibitors are described herein.

One embodiment of the invention is a pharmaceutical composition comprising a pharmaceutically acceptable excipient, carrier or diluent and a compound of Formula I:

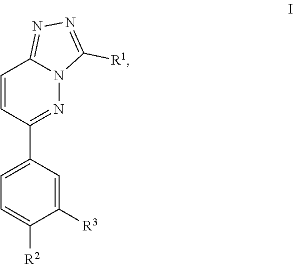

or a pharmaceutically acceptable salt thereof, wherein:
R¹ is C₁₋₃ alkyl, phenyl, or 5-6 membered heteroaryl, wherein R¹ is optionally substituted by halo, —OH, C₁₋₄ alkyl, C₁₋₄ hydroxyalkyl, C₁₋₃ alkoxy, C₁₋₃ haloalkoxy, C₁₋₃ alkylcarbonyl, C₁₋₄ haloalkyl or C₁₋₃ haloalkylcarbonyl;
one of R² and R³ is hydrogen or fluoro, and the other of R² and R³ is —NH(CH₂)ₙCOR⁴ or —NH(CH₂)ₙSO₂R⁴;
n is an integer from zero to three;
R⁴ is phenyl, pyridyl or naphthyl optionally substituted by one, two or three R⁵, or R⁴ is 5-10 membered heteroaryl optionally substituted by one or two R⁵, and
each R⁵ is independently selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CON(C₁₋₄ alkyl)₂, —CO₂(C₁₋₄ alkyl), —NH(C₁₋₄ alkyl), —N(C₁₋₄ alkyl)₂, and C₁₋₄ haloalkoxy.

Another embodiment of the invention is a method of treating cancer in a patient comprising administering to the patient an effective amount of a pharmaceutical composition disclosed herein. Alternatively, the invention is a method of treating cancer in a patient comprising administering to the patient an effective amount of a Rac1 inhibitor disclosed herein or a pharmaceutically acceptable salt thereof.

Another embodiment of the invention is a disclosed Rac1 inhibitors in which one or more hydrogen atoms are replaced by deuterium. Also included are pharmaceutical compositions comprising the deuterated Rac1 inhibitors and methods of treating cancer in a patient by administering to the patient an effective amount of the deuterated Rac1 inhibitors or pharmaceutical compositions comprising the same.

Another embodiment of the invention is a Rac1 inhibitor disclosed in Tables 1-6, or a pharmaceutically acceptable salt thereof.

### DETAILED DESCRIPTION OF THE INVENTION

Disclosed herein are Rac1 inhibitors which can be used in the treatment of a variety of cancers and pharmaceutical compositions comprising the same and a pharmaceutically acceptable excipient, carrier or diluent.

The Rac1 inhibitors used in the disclosed pharmaceutical compositions and methods of treatment are represented by Formula I above or a pharmaceutically acceptable salt thereof.

In a first aspect, the Rac1 inhibitor is represented by Formula I or a pharmaceutically acceptable salt thereof, wherein R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁-C₄ hydroxyalkyl, C₁₋₃ alkylcarbonyl or C₁₋₃ haloalkylcarbonyl; R² is —NH(CH₂)ₙCOR⁴ or —NH(CH₂)ₙSO₂R⁴; R³ is H; R⁴ is phenyl, naphthyl, quinolinyl, pyridyl, furanyl, oxazolyl, thiazolyl, imidazolyl, thienyl or indolyl, each optionally substituted by one or two R⁵; and each R⁵ is independently selected from F, Br, Cl, methyl, ethyl, isopropyl, S-methyl, S-ethyl, S-isopropyl, nitro, methoxy, ethoxy, propoxy and butoxy; and the remainder of the variables are as described for Formula I.

In a second aspect, the Rac1 inhibitor is represented by Formula I or a pharmaceutically acceptable salt thereof, wherein R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁-C₄ hydroxyalkyl, C₁₋₃ alkylcarbonyl or C₁₋₃ haloalkylcarbonyl; R² is —H; R³ is NH(CH₂)ₙCOR⁴ or —NH(CH₂)ₙSO₂R⁴; R⁴ is phenyl, naphthyl, quinolinyl, pyridyl, furanyl or indolyl, each optionally substituted by one or two R⁵; and each R⁵ is independently selected from F, Br, Cl, methyl, ethyl, propyl, S-methyl, S-ethyl, S-isopropyl, nitro, methoxy, ethoxy, propyl and butoxy; and that is a remainder of the variables are as described for Formula I.

In a third aspect, the Rac1 inhibitor is represented by Formula I or a pharmaceutically acceptable salt thereof, wherein R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁₋₄ hydroxyalkyl, C₁₋₃ alkylcarbonyl or C₁₋₃ haloalkylcarbonyl, e.g., C₁₋₄ alkyl or C₁-C₄ hydroxyalkyl; R² is hydrogen; R³ is —NHCOR⁴ or —NHSO₂R⁴; and R⁴ is a phenyl or pyridyl, each optionally substituted with one or two R⁵; and R⁵ is as described for formula I.

In a fourth aspect, the Rac1 inhibitor is represented by Formula I or a pharmaceutically acceptable salt thereof, wherein R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁₋₄ hydroxyalkyl, C₁₋₃ alkylcarbonyl or C₁₋₃ haloalkylcarbonyl, e.g., C₁₋₄ alkyl or C₁-C₄ hydroxyalkyl; R² is —NHCOR⁴ or —NHSO₂R⁴; R³ is hydrogen; and R⁴ is a phenyl or pyridyl, each optionally substituted with one or two R⁵; and R⁵ is as described for Formula I.

In a fifth aspect, the Rac1 inhibitor is represented by Formula I or a pharmaceutically acceptable salt thereof, wherein R⁴ is phenyl substituted by one or two R⁵ independently selected from fluoro, chloro, bromo, —S(methyl), —S(ethyl), —S(isopropyl), methyl, ethyl, trifluoromethyl, methoxy and ethoxy, or R⁴ is a pyridyl optionally substituted substituted by one R⁵ selected from fluoro, chloro, —S(isopropyl), methyl, methoxy or ethoxy; and the remainder of the variables are as described in the third and fourth embodiments or for Formula I.

In a sixth aspect, the Rac1 inhibitor is represented by Formula I or a pharmaceutically acceptable salt thereof, wherein R¹ is methyl, 3-fluorophenyl, 5-methyl-furan-2-yl, thiazol-5-yl, or an unsubstituted pyridyl; R² is hydrogen; R³ is —NHCOR⁴; and R⁴ is 3-fluorophenyl, 3-chlorophenyl, or 3-S(isopropyl)phenyl; and R⁵ is as described for Formula I.

In a seventh aspect, the Rac1 inhibitor is represented by Formula I or a pharmaceutically acceptable salt thereof, wherein R¹ is pyridyl, 5-methyl-furan-2-yl, thiazol-5-yl; one of R² or R³ is —H and the other is —NHSO₂R⁴ or —NHCOR⁴; R⁴ is phenyl, indolyl or pyridyl optionally substituted with one or two R⁵; and each R⁵ is independently selected from fluoro, methyl, methoxy and ethoxy.

In an eight aspect, the Rac1 inhibitor is represented by Formula I or a pharmaceutically acceptable salt thereof, wherein each R⁵ is independently selected from R⁵ is independently selected from F, Br, Cl, —S(isopropyl), methyl, ethyl, propyl, isobutyl, nitro, methoxy, ethoxy, propoxy and butoxy; and the remainder of the variables are as described in the third, fourth, and sixth aspects.

In a ninth aspect, the Rac1 inhibitor is represented by Formula I or a pharmaceutically acceptable salt thereof, wherein R¹ is pyridyl, oxazolyl, thiazolyl, furanyl, tetrahydrofuranyl, pyrrolyl, pyrazolyl or thienyl, e.g., pyridyl, thiazolyl, furanyl, or thienyl, and R¹ is optionally substituted by C₁₋₄ alkyl, C₁₋₄ hydroxyalkyl, C₁₋₃ alkoxy, C₁₋₃ haloalkoxy, C₁₋₃ alkylcarbonyl, haloalkyl or haloalkylcarbonyl; and the remainder of the variables are as described for formula I or the first or second aspects.

In a tenth aspect, the Rac1 inhibitor is represented by Formula I or a pharmaceutically acceptable salt thereof, wherein R¹ is one of the following:

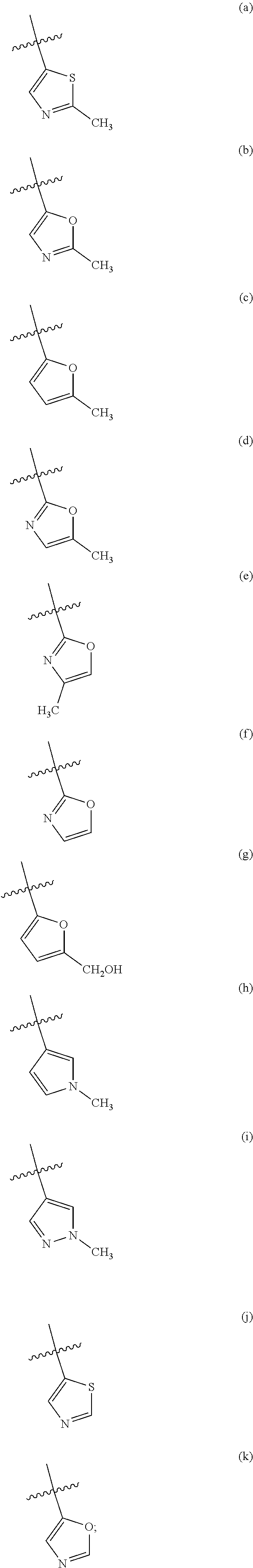

and the remainder of the variables are as described for Formula I or the first, second, third, fourth or fifth aspect.

In an eleventh aspect, the Rac1 inhibitors are represented by Formulas I-A-1, I-B-1, I-A-2 and I-B-2:

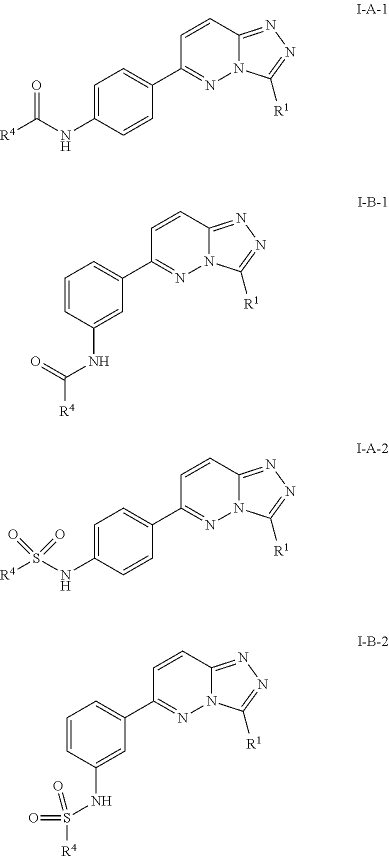

or a pharmaceutically acceptable salt thereof, wherein R¹ and R⁴ are as described for Formula I. Alternatively, R¹ and R⁴ are as described in any one of the first, second, third, fourth or fifth or tenth aspects.

In a twelfth aspect, the Rac1 inhibitor is represented by Formula I where there is a first R⁵ selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy and optionally a second R⁵ selected from halo, C₁₋₅ alkyl, C₁₋₅ haloalkyl, or C₁₋₄ alkoxy. Typically, the first R⁵ is substituted at the meta position of an R⁴ phenyl ring and the second R⁵, if present, is substituted in the para position of the phenyl ring. The remainder of the variables are as described for Formula I or any one of the first, second, third or fourth aspects.

In a thirteenth aspect, the Rac1 inhibitor is a compound of Formula I-B-1:

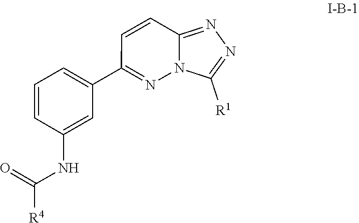

or a pharmaceutically acceptable salt thereof, wherein:
R¹ is a 5-6 membered heteroaryl ring (e.g., pyridyl, furanyl, thiazolyl, pyrrolyl, pyrazolyl or oxazolyl; or a formula selected from Formulas (a) through (k) above) optionally substituted by C₁₋₄ alkyl, C₁₋₄ hydroxyalkyl, C₁₋₃ alkoxy, C₁₋₃ haloalkoxy, C₁₋₃ alkylcarbonyl, haloalkyl or haloalkylcarbonyl;
R⁴ is phenyl or pyridyl substituted by one or two R⁵; and
each R⁵ is independently selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy.

Alternatively, the Rac1 inhibitor is represented by Formula I-B-1(a), or a pharmaceutically acceptable salt thereof, there is a first R⁵ selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy and optionally a second R⁵ selected from halo, C₁₋₅ alkyl, C₁₋₅ haloalkyl, or C₁₋₄ alkoxy. Alternatively R⁴ is phenyl substituted in the meta-position by the first R⁵ and optionally substituted in the para-position by the second R⁵. The remainder of the variables for both alternatives are as described in the thirteenth aspect.

In a fourteenth aspect, the Rac1 inhibitor is represented by Formula I-B-1(a):

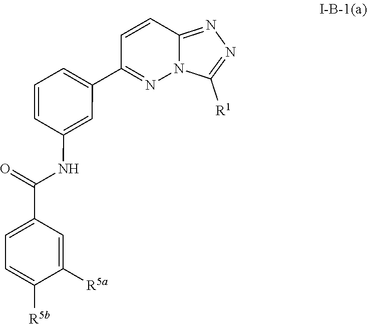

or a pharmaceutically acceptable salt thereof, where R^{5a} is a first R⁵ substituent selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy and R^{5b} is optional. R^{5b}, when present, is selected from halo, C₁₋₅ alkyl, and C₁₋₄ alkoxy. Particular examples of R^{5a} include fluoro, chloro, bromo, ethyl, isobutyl, S(isopropyl), methoxy, and ethoxy. Particular examples of R^{5b} include fluoro and methyl. With respect to the substitution position of the R⁴ phenyl ring, R^{5a} is referred to as being in the meta position and R^{5b} in the para position. R¹ is as described for Formula I or in the first, second, third, fourth or tenth aspects.

In a fifteenth aspect, the Rac1 inhibitor is represented by Formula (I) or a pharmaceutically acceptable salt thereof, wherein R¹ is C₁₋₃ alkyl, phenyl, or 5-6 membered heteroaryl, wherein R¹ is optionally substituted by halo, —OH or C₁₋₃ alkoxy; one of R² and R³ is hydrogen or fluoro, and the other of R² and R³ is —NH(CH₂)ₙCOR⁴ or —NH(CH₂)ₙSO₂R⁴; n is an integer from zero to three; R⁴ is phenyl or naphthyl optionally substituted by one, two or three R⁵, or R⁴ is 5-10 membered heteroaryl optionally substituted by one or two R⁵, each R⁵ is independently selected from halo, C₁₋₄ alkyl, C₁₋₄ haloalkyl, C₁₋₄ alkoxy and C₁₋₄ haloalkoxy and the remainder of the variables are as described for Formula (I).

In a sixteenth aspect, the Rac1 inhibitor is represented by Formula I or a pharmaceutically acceptable salt thereof, wherein R¹ is methyl, phenyl, pyridyl or thienyl, e.g., methyl, pyridyl or thienyl; R² is —NH(CH₂)ₙCOR⁴ or —NH(CH₂)ₙSO₂R⁴; R³ is H; R⁴ is phenyl, naphthyl, quinolinyl, pyridyl, furanyl or indolyl, each optionally substituted by one or two R⁵; each R⁵ is independently selected from F, Br, Cl, methyl, ethyl, nitro, methoxy, ethoxy, and butoxy; and the remainder of the variables are as described in the fifteenth aspect.

In a seventeenth aspect, the Rac1 inhibitor is represented by Formula I or a pharmaceutically acceptable salt thereof, wherein R¹ is methyl, phenyl, pyridyl or thienyl, e.g., methyl, pyridyl or thienyl; R² is —H; R³ is NH(CH₂)ₙCOR⁴ or —NH(CH₂)ₙSO₂R⁴; R⁴ is phenyl, naphthyl, quinolinyl, pyridyl, furanyl or indolyl, each optionally substituted by one or two R⁵; each R⁵ is independently selected from F, Br, Cl, methyl, ethyl, nitro, methoxy, ethoxy, and butoxy; and the remainder of the variables are as described in the fifteenth aspect.

In an eighteenth aspect, the Rac1 inhibitor is represented by Formula I or a pharmaceutically acceptable salt thereof, where R¹ is methyl, phenyl, thienyl, or pyridyl, e.g., methyl, thienyl or pyridyl; R² is hydrogen; R³ is —NHCOR⁴ or —NHSO₂R⁴; R⁴ is a phenyl or pyridyl, each optionally substituted with one or two R⁵; and R⁵ is as described in the fifteenth aspect.

In a nineteenth aspect, the Rac1 inhibitor is represented by Formula I or a pharmaceutically acceptable salt thereof, wherein R¹ is methyl, phenyl, thienyl, or pyridyl e.g., methyl, thienyl or pyridyl; R² is —NHCOR⁴ or —NHSO₂R⁴; R³ is hydrogen; R⁴ is a phenyl or pyridyl, each optionally substituted with one or two R⁵; and R⁵ is as described in the fifteenth aspect.

In a twentieth aspect, the Rac1 inhibitor is represented by Formula I or a pharmaceutically acceptable salt thereof, wherein R¹ is methyl, phenyl, or pyridyl, e.g., methyl or pyridyl; and R⁴ is phenyl substituted by one or two R⁵ independently selected from fluoro, chloro, methyl, ethyl, trifluoromethyl, methoxy and ethoxy, or R⁴ is a pyridyl optionally substituted by one R⁵ selected from fluoro, chloro, methyl, methoxy or ethoxy; and the remainder of the variables are as described in the eighteenth or nineteenth embodiments.

In a twenty first aspect, the Rac1 inhibitor is represented by Formula I or a pharmaceutically acceptable salt thereof, wherein R¹ is methyl, 3-fluorophenyl or an unsubstituted pyridyl; R² is hydrogen; R³ is —NHCOR⁴; and R⁴ is 3-fluorophenyl.

In a twenty second aspect, the Rac1 inhibitor is represented by Formula I or a pharmaceutically acceptable salt thereof, wherein R¹ is pyridyl; one of R² or R³ is —H and the other is —NHSO₂R⁴ or —NHCOR⁴; R⁴ is phenyl, indolyl or pyridyl optionally substituted with one or two R⁵; and each R⁵ is independently selected from fluoro, methyl, methoxy and ethoxy.

In a twenty third aspect, the Rac1 inhibitor is represented by Formula I or a pharmaceutically acceptable salt thereof, wherein each R⁵ is independently selected from F, Br, Cl, methyl, ethyl, nitro, methoxy, ethoxy and butoxy; and the remainder of the variables are as described in the eighteenth and nineteenth aspects.

In a twenty fourth aspect, the Rac1 inhibitor is represented by one of Formulas I-A-1, I-B-1, I-A-2 and I-B-2, or a pharmaceutically acceptable salt thereof, wherein herein R¹ and R⁴ are as described for the fifteenth aspect. Alternatively, R¹ and R⁴ are as described in any one of the sixteenth through twenty first aspects.

The Rac1 inhibitors disclosed in Tables 1-6 below and pharmaceutically acceptable salts thereof can also be used in the disclosed pharmaceutical formulations and methods.

TABLE 1
Examples of Compounds of Formula I-A-1
I-A-1

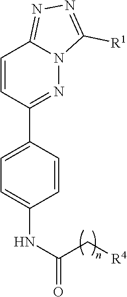

| Compound | | | | |
|---|---|---|---|---|
| No. | R¹ | R⁴ | n | IC₅₀ (uM) |
| 1 | 2-pyridyl | 3-fluorophenyl | 0 | ††† |
| 2 | 3-pyridyl | 3-fluorophenyl | 0 | NT |
| 3 | 2-pyridyl | 3-methylphenyl | 0 | ††† |
| 4 | 2-pyridyl | 3,4-dimethylphenyl | 0 | † |
| 5 | 2-pyridyl | 2-chlorophenyl | 0 | ††† |
| 6 | 2-pyridyl | 3,5-dimethylphenyl | 0 | †† |
| 7 | 4-pyridyl | 4-chlorophenyl | 0 | † |
| 8 | 4-pyridyl | 3-fluorophenyl | 0 | † |
| 9 | 4-pyridyl | 3,4-dimethylphenyl | 0 | †† |
| 10 | 4-pyridyl | 3-chlorophenyl | 0 | ††† |
| 11 | 4-pyridyl | 3,5-dimethylphenyl | 0 | †† |
| 12 | 4-pyridyl | 4-fluorophenyl | 0 | ††† |
| 13 | 4-pyridyl | 2-methylphenyl | 0 | †† |
| 14 | 4-pyridyl | 2-chlorophenyl | 0 | †† |
| 15 | 4-pyridyl | naphth-1-yl | 0 | ††† |
| 16 | 4-pyridyl | naphth-2-yl | 0 | †† |
| 17 | 4-pyridyl | 3-nitrophenyl | 0 | †† |
| 18 | 3-pyridyl | thien-2-yl | 1 | †† |
| 19 | 2-pyridyl | naphth-1-yl | 0 | ††† |
| 20 | 2-pyridyl | quinoline-2-yl | 0 | † |
| 21 | 2-pyridyl | 2-chloro-pyrid-5-yl | 0 | †† |
| 22 | 2-pyridyl | 2-chloro-pyrid-3-yl | 0 | ††† |
| 23 | 2-pyridyl | 2-methyl-phenyl | 0 | ††† |
| 24 | 2-thienyl | 3-fluorophenyl | 0 | ††† |
| 25 | 2-thienyl | 3-methoxyphenyl | 0 | †† |
| 26 | 2-thienyl | 4-fluorophenyl | 0 | ††† |
| 27 | 2-thienyl | 2,4-difluorophenyl | 0 | ††† |
| 28 | 2-thienyl | 2-chlorophenyl | 0 | ††† |
| 29 | CH₃ | 2,3- | 0 | † |
| | | dimethoxyphenyl | | |
| 30 | 2-pyridyl | phenyl | 1 | ††† |
| 31 | CH₃ | 2-furanyl | 0 | † |

TABLE 2
Examples of Compounds of Formula I-A-2
I-A-2

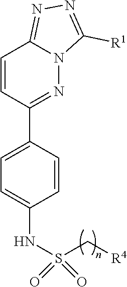

| Compound | | | | |
|---|---|---|---|---|
| No. | R¹ | R⁴ | n | IC₅₀ (uM) |
| 32 | 2-pyridyl | 4-methylphenyl | 0 | ††† |
| 33 | 2-pyridyl | phenyl | 0 | ††† |
| 34 | 2-pyridyl | 3-methylphenyl | 0 | ††† |

TABLE 3
Examples of Compounds of Formula I-B-1
I-B-1

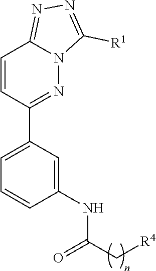

| Compound | | | | |
|---|---|---|---|---|
| No. | R¹ | R⁴ | n | IC₅₀ (uM) |
| 35 | 2-pyridyl | 3-fluorophenyl | 0 | ††† |
| 36 | 3-pyridyl | 3-fluorophenyl | 0 | ††† |
| 37 | 3-pyridyl | 3-methylphenyl | 0 | ††† |
| 38 | 3-pyridyl | 3,5-dimethylphenyl | 0 | ††† |
| 39 | 3-pyridyl | 4-methoxyphenyl | 0 | ††† |
| 40 | 3-pyridyl | 4-ethoxyphenyl | 0 | †† |
| 41 | 3-pyridyl | 2,4-difluorophenyl | 0 | 14% POC |
| 42 | 4-pyridyl | 3-fluorophenyl | 0 | ††† |
| 43 | CH₃ | 3-bromophenyl | 0 | †† |
| 44 | 3-pyridyl | 2-chlorophenyl | 1 | ††† |
| 45 | 3-pyridyl | indol-3-yl | 1 | ††† |
| 46 | 3-pyridyl | 3-methoxyphenyl | 1 | ††† |
| 47 | 3-pyridyl | 2-nitrophenyl | 0 | ††† |
| 48 | 3-pyridyl | naphth-1-yl | 0 | ††† |
| 49 | 3-pyridyl | naphtha-2-yl | 0 | †† |
| 50 | 3-pyridyl | quinolin-2-yl | 0 | †† |
| 51 | 3-pyridyl | thien-2-yl | 1 | ††† |
| 52 | 3-pyridyl | 4-chlorophenyl | 1 | ††† |
| 53 | 3-pyridyl | 3-nitrophenyl | 0 | †† |
| 54 | CH₃ | 2-ethoxy-pyrid-3-yl | 0 | ††† |
| 55 | 3-pyridyl | phenyl | 3 | ††† |
| 64 | 3-pyridyl | 3-methoxyphenyl | 0 | ††† |
| 65 | 3-pyridyl | 4-fluorophenyl | 0 | ††† |
| 66 | 3-pyridyl | 2-bromophenyl | 0 | ††† |
| 67 | 3-pyridyl | 2-methoxyphenyl | 0 | ††† |
| 68 | 3-pyridyl | 3-chlorophenyl | 0 | ††† |
| 69 | 3-pyridyl | thiazol-5-yl | 0 | ††† |
| 70 | 3-pyridyl | oxazol-5-yl | 0 | ††† |
| 71 | 3-pyridyl | 2-ethoxy-pyridin-3-yl | 0 | ††† |
| 72 | 3-pyridyl | pyridin-3-yl | 0 | ††† |
| 73 | 3-pyridyl | pyridin-2-yl | 0 | ††† |
| 74 | 3-pyridyl | (CH₂)—N-methyl-indol-3-yl | 0 | ††† |
| 75 | 3-pyridyl | N-methyl-imidazol-5-yl | 0 | ††† |
| 76 | 3-pyridyl | 2,5-diethoxy-pyridin-4-yl | 0 | ††† |
| 77 | 3-pyridyl | 2-ethoxy-pyridin-4-yl | 0 | ††† |
| 78 | 3-pyridyl | 4-fluoro-3-methoxyphenyl | 0 | ††† |

TABLE 4
Examples of Compounds of Formula I-B-2
I-B-2

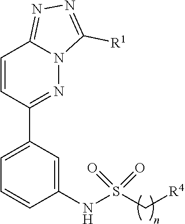

| Compound | | | | |
|---|---|---|---|---|
| No. | R¹ | R⁴ | n | IC₅₀ (uM) |
| 56 | 3-pyridyl | 2,4-difluorophenyl | 0 | ††† |
| 57 | 3-pyridyl | naphth-2-yl | 0 | ††† |
| 58 | 3-pyridyl | 4-butoxy-phenyl | 0 | ††† |
| 59 | 3-pyridyl | 5-ethyl-thiophen-2-yl | 0 | ††† |
| 60 | 3-pyridyl | 5-methyl-thiophen-2-yl | 0 | ††† |
| 61 | 3-pyridyl | 3,5-dimethylphenyl | 0 | ††† |
| 62 | 3-pyridyl | 2-fluorophenyl | 0 | ††† |
| 63 | 3-pyridyl | 3-fluorophenyl | 0 | ††† |
| 106 | 5-methylfuran-2-yl | 2,4-difluorophenyl | 0 | ††† |
| 107 | 3-pyridyl | 4-methoxyphenyl | 0 | ††† |
| 108 | 3-pyridyl | 2,4-dimethoxyphenyl | 0 | ††† |

TABLE 5
Examples of Compounds of Formula I-B-1 (where R¹ is 2-furanyl):

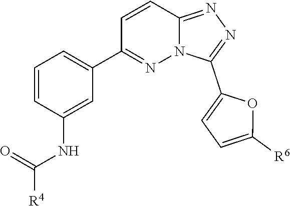

| Compound | | | |
|---|---|---|---|
| No. | R⁴ | R⁶ | IC₅₀ (uM) |
| 79 | 3-methoxyphenyl | CH₃ | ††† |
| 80 | 4-fluorophenyl | CH₃ | ††† |
| 81 | 3-chloro-4-fluorophenyl | CH₃ | ††† |
| 82 | 3-chlorophenyl | CH₃ | ††† |
| 83 | 3-bromophenyl | CH₃ | ††† |
| 84 | 3-fluorophenyl | CH₃ | ††† |
| 85 | 3,4-difluorophenyl | CH₃ | ††† |
| 86 | 4-F-3-SCH₃phenyl | CH3 | ††† |
| 87 | 3-SCH₂CH₃phenyl | CH₃ | ††† |
| 88 | 3-SCH(CH₃)₂phenyl | CH3 | ††† |
| 89 | 3-methylphenyl | CH₃ | ††† |
| 90 | 3-ethylphenyl | CH₃ | ††† |
| 91 | 4-F-3-SCH(CH₃)₂phenyl | CH3 | ††† |
| 92 | 4-fluoro-3-methylphenyl | CH₃ | ††† |
| 93 | 3-ethyl-4-fluorophenyl | CH3 | ††† |
| 94 | 3-bromo-4-fluorophenyl | CH₃ | ††† |
| 95 | 4-fluorophenylphenyl | CH₂OH | ††† |
| 96 | 3-CH₂CH(CH₃)₂-phenyl | CH₃ | NA |
| 97 | 3-CH₂CH(CH₃)₂-4-fluorophenyl | CH₃ | NA |
| 105 | 3-(SO₂CH(CH₃)₂)-phenyl | CH₃ | NA |
| 109 | 5-oxazolyl | CH₃ | ††† |
| 110 | 3-CH₂CH₂CH(CH₃)₂-4-fluorophenyl | CH₃ | ††† |
| 111 | 3-S(tBu)-phenyl | CH₃ | ††† |
| 112 | 4-fluorophenylphenyl | CHO | ††† |
| 113 | 4-fluorophenylphenyl | CO₂CH₃ | NA |

TABLE 6
Examples of Compounds of Formula I-B-1 (where R¹ is thiazol-5-yl):

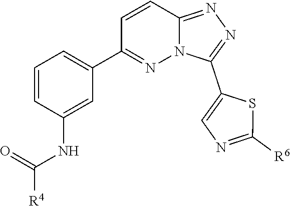

| Compound | | | |
|---|---|---|---|
| No. | R⁴ | R⁶ | IC₅₀ (uM) |
| 98 | 3-methoxyphenyl | H | ††† |
| 99 | 4-fluorophenyl | H | ††† |
| 100 | 3-chlorophenyl | H | — |
| 101 | 3-chloro-4-fluorophenyl | H | — |
| 102 | 3-ethylphenyl | H | |
| 103 | 3-ethyl-4-fluorophenyl | H | |
| 104 | 3-S(isopropyl)phenyl | H | |

The IC₅₀ values reported in Tables 1-6 were obtained from the Rae Activation AlphaScreen Assay (Rac1 AS) assay described in subsection 2 under the section entitled “In Vitro Assays”. “†” represents an IC₅₀ of greater than 100 uM; “††” represents an IC₅₀ of greater than 10 uM and less than 50 uM; and “†††” represents an IC₅₀ of less than 10 uM. “POC” means percent of control. POC shows the percent of Rac1 activation at 10 uM of inhibitor relative to a control which lacks an inhibitor. Lower POC values reflect greater inhibition. “NT” means not tested.

Another embodiment of the invention is a disclosed Rac1 inhibitor in which one or more hydrogen atoms are replaced by deuterium. When a hydrogen atom is replaced by deuterium at a particular position, the position is understood to have deuterium at an abundance that is at least 3000 times greater than the natural abundance of deuterium, which is 0.015% (i.e., at least 45% incorporation of deuterium). Alternatively, the deuterium incorporation is at least 52.5% at each designated position, at least 60% at each designated position, at least 67.5% at each designated position, at least 75% at each designated position, at least 82.5% at each designated position at least 90% at each designated position, at least 95% at each designated position, at least 97% at each designated position, at least 99% at each designated position, or at least 99.5% at each designated position.

In one aspect, R¹ and/or R⁵ of the disclosed Rac1 inhibitors are deuterated when R¹ and/or R⁵ are alkyl, alkoxy or have an alkyl or alkoxy moiety. In another aspect, the alkyl and/or alkoxy group represented by R¹ and/or R⁵ are perdeuterated, i.e., all of the hydrogen atoms are replaced with deuterium. In another aspect, the deuterated groups represented by R¹ and/or R⁵ are methyl or methoxy, and in another aspect, these groups are perdeuterated.

In another aspect, each alkylene carbon is independently substituted with 0 or 2 deuterium. —(CH₂)ₙ— is an alkylene group. In yet another aspect, R¹ and R⁵ are as just described in the preceding paragraph and each alkylene carbon is independently substituted with 0 or 2 deuterium.

When a position is designated specifically as “H” or “hydrogen”, the position is understood to have hydrogen at its natural abundance isotopic composition. When a position is designated specifically as “D” or “deuterium”, the position is understood to be enriched in deuterium, as described above. When there is no specific designation as to whether a position has hydrogen or deuterium, it is understood that the position has hydrogen at natural abundance. For example, the term “methyl”, unless there is a specific designation to the contrary, is understood to mean —CH₃ with all three hydrogen atoms present at natural abundance, and the term “phenyl”, unless there is a specific designation to the contrary, means all five hydrogen atoms to be present at natural abundance.

As used herein, “alkyl” refers to a fully saturated branched or unbranched hydrocarbon moiety. Unless otherwise specified, an alkyl comprises 1 to 4 carbon atoms. Representative examples of alkyl include, but are not limited to, methyl, ethyl, n-propyl, iso-propyl, n-butyl, sec-butyl, iso-butyl and tert-butyl.

“Halogen” or “halo” may be fluoro, chloro, bromo or iodo.

As used herein, the term “heteroaryl” refers to an aromatic 5- to 6-membered monocyclic or a 7- to 10-membered bicyclic ring system, having 1 to 4 heteroatoms independently selected from O, N and S, and wherein N can be oxidized (e.g., N(O)) or quaternized, and S can be optionally oxidized to sulfoxide and sulfone. Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, pyridazinyl, triazinyl, tetrazinyl, and the like. Examples of 8- to 10-membered bicyclic heteroaryls include, but are not limited to, dihydropyrrolopyrrolyl, indolyl, isoindolyl, benzimidazolyl, benzothiazolyl, quinolinyl, isoquinolinyl, quinazolinyl and purinyl.

In many cases, the compounds of the present invention are capable of forming acid and/or base salts by virtue of the presence of amino and/or carboxyl groups or groups similar thereto. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable.

Pharmaceutically acceptable acid addition salts can be formed with inorganic acids and organic acids, e.g., tosylate, methanesulfonate, acetate, citrate, malonate, tartarate, succinate, benzoate, hydrochloride, sulfate, nitrate, bicarbonate, phosphate and carbonate salts. Lists of additional suitable salts can be found, e.g., in REMINGTON'S PHARMACEUTICAL SCIENCES, 20th ed., Mack Publishing Company, Easton, Pa., (1985); and in HANDBOOK OF PHARMACEUTICAL SALTS: PROPERTIES, SELECTION, AND USE, by Stahl and Wermuth (Wiley-VCH, Weinheim, Germany. 2002).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, REMINGTON'S PHARMACEUTICAL SCIENCES, 18th Ed. Mack Printing Company, 1990, pp. 1289-1329). Except insofar as any conventional carrier is incompatible with the active ingredient, its use in the therapeutic or pharmaceutical compositions is contemplated.

The term “effective amount” (used interchangeably with “therapeutically effective amount”) of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or reduce the likelihood or delay reoccurrence of a disease, etc. In one non-limiting embodiment, the term “a therapeutically effective amount” refers to the amount of the compound of the present invention that, when administered to a subject, is effective to (1) at least partially alleviate, inhibit and/or ameliorate a condition, or a disorder or a disease (i) mediated by hyperactivation of Rac1 or (ii) associated with overexpression of Rac1, or (iii) characterized by activity (normal or abnormal) of Rac1.

In another non-limiting embodiment, the term “a therapeutically effective amount” or “an effective amount” refers to the amount of the compound of the present invention that, when administered to a cell, or a tissue, or a non-cellular biological material, or a medium, is effective to at least partially reduce or inhibit the activity of Rac1, or at least partially reduce or inhibit the expression of Rac1.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or “inhibiting” refers to the reduction or suppression of a given condition, activity, effect, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to delaying the progression of the disease or disorder. In another embodiment, “treat”, “treating” or “treatment” refers to reducing the likelihood or delaying reoccurrence of the disease or disorder after it has gone into remission.

The pharmaceutical composition or combination of the present invention can be in unit dosage of about 1-2000 mg of active ingredient(s) for a subject of about 50-70 kg, of active ingredients. The therapeutically effective dosage of a compound, the pharmaceutical composition, or the combinations thereof, is dependent on the species of the subject, the body weight, age and individual condition, the disorder or disease being treated and the severity thereof. A physician, clinician or veterinarian of ordinary skill can readily determine the effective amount of each of the active ingredients necessary to prevent, treat or inhibit the progress of the disorder or disease.

As indicated above, a further embodiment of the invention relates to a pharmaceutical composition comprising at least one compound of the invention and a pharmaceutically acceptable diluent, excipient, or carrier.

The compounds of the invention are typically administered with suitable pharmaceutical diluents, excipients, or carriers (collectively referred to herein as pharmaceutically acceptable carriers). Suitable pharmaceutical diluents, excipients, and carriers include, but are not limited to, lubricants, solvents, binders, and stabilizers that are suitably selected with respect to the intended form of administration including solid and liquid forms, such as capsules, tablets, gels, solutions, syrups, suspensions, powders, aerosols, ointments, and the like.

Diluents that may be used in the compositions of the invention include but are not limited to dicalcium phosphate, calcium sulfate, lactose, cellulose, kaolin, mannitol, sodium chloride, dry starch, powdered sugar and hydroxy propyl methyl cellulose (HPMC). The binders that may be used in the compositions of the invention include but are not limited to starch and gelatin. Additionally, fillers such as sucrose, glucose, dextrose and lactose may also be used.

Natural and synthetic gums that may be used in the compositions of the invention include but are not limited to sodium alginate, ghatti gum, carboxymethyl cellulose, methyl cellulose, polyvinyl pyrrolidone and veegum. Excipients that may be used in the compositions of the invention include but are not limited to microcrystalline cellulose, calcium sulfate, dicalcium phosphate, starch, magnesium stearate, lactose, and sucrose. Stabilizers that may be used in the compositions of the invention include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used in the composition of the invention include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds of the invention are administered will vary according to the dosage form, mode of administration, the condition being treated and particulars of the patient being treated. Accordingly, optimal therapeutic concentrations will be best determined at the time and place through routine experimentation.

The compounds according to the invention can also be used enterally. Orally, the compounds according to the invention are suitably administered at the rate of 10 μg to 300 mg per day per kg of body weight. The required dose can be administered in one or more portions. For oral administration, suitable forms are, for example, capsules, tablets, gels, aerosols, pills, dragees, syrups, suspensions, emulsions, solutions, powders and granules. A preferred method of administration consists of using a suitable form containing from 0.01 mg to about 500 mg of active substance.

The compounds according to the invention can also be administered parenterally in the form of solutions or suspensions for intravenous, subcutaneous or intramuscular perfusions or injections. In that case, the compounds according to the invention are generally administered at the rate of about 10 μg to 10 mg per day per kg of body weight. A preferred method of administration consists of using solutions or suspensions containing approximately from 0.01 mg to 1 mg of active substance per ml.

The compounds may be administered according to various routes, typically by oral route or by injection, such as local or systemic injection(s). Intratumoral injections are preferred for treating existing cancers. However, other administration routes may be used as well, such as intramuscular, intravenous, intradermic, subcutaneous, etc. Furthermore, repeated injections may be performed, if needed, although it is believed that a limited number of injections will be needed in view of the efficacy of the compounds.

The compounds of the invention can be used in a substantially similar manner to other known anti-tumor agents for treating (both chemopreventively and therapeutically) various tumors. The dose to be administered, whether a single dose, multiple dose, or a daily dose, will vary with the particular compound employed because of the varying potency of the compound, the chosen route of administration, the size of the recipient, the type of disease, and the nature of the patient's condition. The dosage to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. An oncologist skilled in the art of cancer treatment or a doctor skilled in the art in treating kidney or heart disease will be able to ascertain, without undue experimentation, appropriate protocols for the effective administration of the compounds of this present invention.

The compounds of the invention may also be administered in combination with other known therapies. For example, the compounds of the invention can be administered in combination with other known chemotherapy drugs. When co-administered with one or more other therapies, the compounds of the invention can be administered either simultaneously with the other treatment(s), or sequentially. If administered sequentially, the attending physician will decide on the appropriate sequence of administering the compounds of the invention in combination with the other therapy.

### Synthesis of Compounds

Compounds of the invention can be prepared according to the Scheme below.

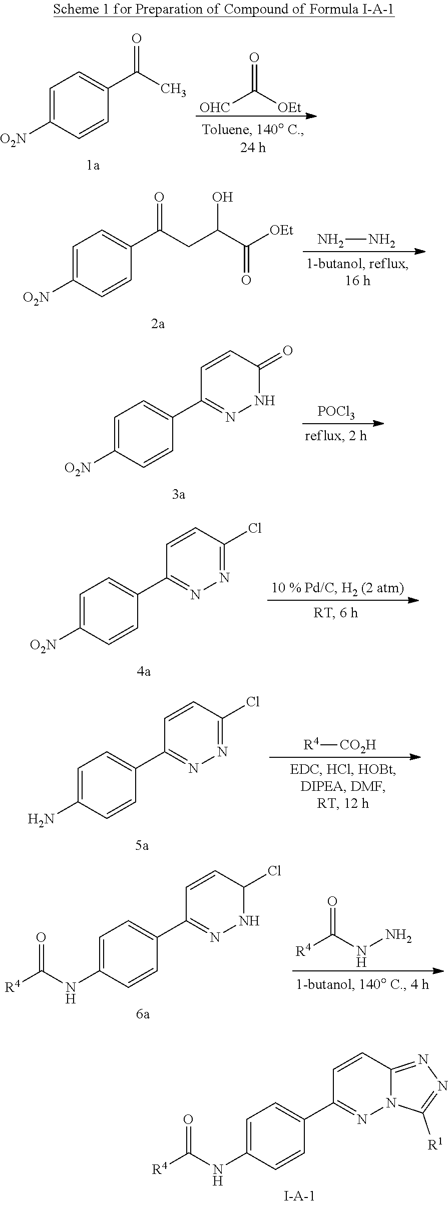

Compounds of Formula I-A-1 may be prepared according to the Scheme in a manner analogous to methods described in patent application WO2008/030344A2. Para-nitroacetophenone (1a) is heated in toluene with ethyl glyoxalate to provide ethyl β-(4-nitrobenzoyl)lactate (2a). A solution of 2a and hydrazine hydrate in 1-butanol is mixed at elevated temperature to provide the pyridazinone 3a. Chlorination of 3a with POCl₃ yields 3-chloro-6-(4-nitrophenyl)pyridazine (4a). The nitro group of 4a is reduced with palladium on charcoal to provide 3-chloro-6-(4-aminophenyl)pyridazine (5a). A solution of 5a and is coupled with the appropriate carboxylic acid (R⁴—CO₂H) to obtain the corresponding carboxamide 6a. The desired compound I-A-1 is prepared by heating a solution of 6a with the appropriate hydrazide (R¹—CONHNH₂).

In a manner analogous to that shown in Scheme I above, sulfonamide compounds I-A-2 where R² is —NHSO₂R⁴ may be prepared by coupling intermediate 5a with the appropriate sulfonyl chloride (R⁴—SO₂Cl).

In a manner analogous to that shown in the Scheme above, compounds I-B-1 may be prepared by starting with a meta-nitrophenone in place of para-nitrophenone (1a). Compounds of formula I-B-1 where R¹ is a 5-membered heteroaryl such as 2-furanyl were prepared as shown in the Scheme below.

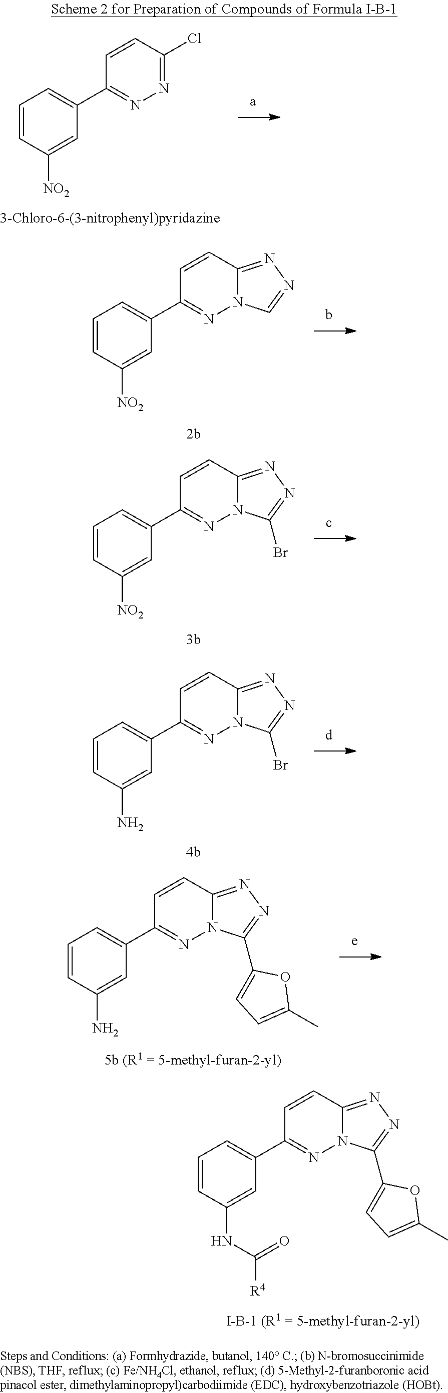

Compounds of Formula I-B-1 were prepared according to Scheme 2. The known 3-chloro-6-(3-nitrophenyl)pyridazine, which is also commercially available, was condensed with fornhydrazide according to step (a) to provide the triazolopyridazine 2b. Bromination with NBS gave the bromo intermediate 3b, which was reduced to the amine 4b as shown in step (c). Suzuki-type coupling with 5-methyl-2-furanboronic acid pinacol ester provided 5b. Intermediate 5b was generally useful for preparing compounds of Formula I-B-1 where R¹ is 5-methyl-furan-2-yl employing standard amide coupling conditions as shown in step (e). Scheme 2 may be readily adapted to prepare other compounds of Formula I-B-1 having other R¹ substituents that are 5-6 membered heteroaryl or aryl rings by using the boronic ester of the desired R¹ substituent.

Standard coupling reactions were useful in preparing a number of compounds of the invention having various R¹ groups, examples of which are shown in Table 7 below.

TABLE 7
Examples of Coupling Reactions for Preparing Compounds of Formula I-B-1
Having Various R¹ Groups

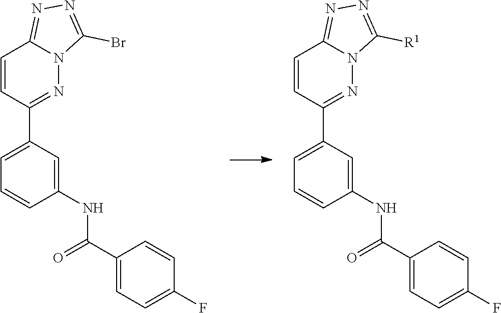

| Compound | | | |
|---|---|---|---|
| No. | Coupling Partner | Reaction Conditions | R¹ |
| 95 | 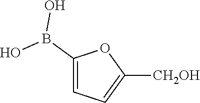 | PdCl₂(dppf), K₂CO₃, dioxane, 110° C., 12h | 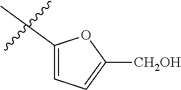 [SMILES: *c1ccc(CO)o1] |
| 99 | 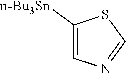 | PdCl₂(dppf), dioxane, 140° C. | 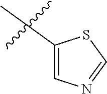 [SMILES: *c1cncs1] |
| 112 | 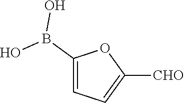 | PdCl₂(PPh₃)₂, CsCO₃, dioxane, 110° C., 12h | 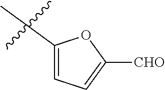 [SMILES: *c1ccc(C=O)o1] |
| 113 | 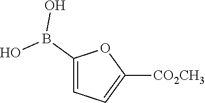 [SMILES: COC(=O)c1ccc(B(O)O)o1] | PdCl₂(PPh₃)₂, CsCO₃, dioxane, 110° C., 12h | 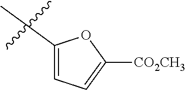 [SMILES: *c1ccc(C(=O)OC)o1] |

A number of disclosed Rac1 inhibitors where R¹ is unsubstituted pyridyl or phenyl can also be purchased from Life Chemicals (https://lifechemicals.com/) located at 1a Dixie Avenue, PO Box 229, Niagara-on-the-Lake, Ontario, Canada LOS 1J0. Compounds of Formula I where R¹ is (substituted)thienyl or an optionally substituted oxazolyl, thiazolyl, furanyl, tetrahydofuranyl, pyrrolyl, and pyrazolyl are new. As such, the invention is directed to a compound of formula I, wherein R¹ is (substituted)thienyl or an optionally substituted oxazolyl, thiazolyl, furanyl, tetrahydofuranyl, pyrrolyl, and pyrazolyl, and the remainder of the variables are as described in aspect one through five and eleven through fourteen.

Another embodiment of the invention provides a compound of Formula II:

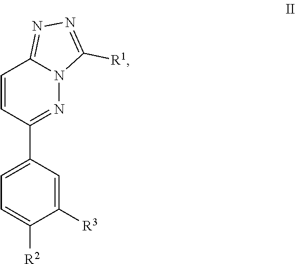

or a pharmaceutically acceptable salt thereof, wherein
R¹ is a substituted 5-6 membered heteroaryl;
one of R² and R³ is hydrogen or fluoro, and the other of R² and R³ is —NH(CH₂)ₙCOR⁴ or —NH(CH₂)ₙSO₂R⁴;
n is an integer from zero to three;
R⁴ is phenyl or naphthyl optionally substituted by one, two or three R⁵, or R⁴ is 5-10 membered heteroaryl optionally substituted by one or two R⁵, and
each R⁵ is independently selected from halo, C₁₋₅ alkyl, —S(C₁₋₄ alkyl), C₁₋₄ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy.

In a second compound embodiment, the invention relates to compounds of Formula II or a pharmaceutically acceptable salt thereof, wherein R¹ is: i) substituted pyridyl or thienyl; or ii) optionally substituted furanyl, oxazolyl, thienyl, thiazolyl, pyrrolyl, or pyrazolyl, wherein the remainder of the variables are as described for Formula II. Examples of substituents on R¹ include halo, C₁₋₄ alkyl, —OH, C₁₋₃ alkoxy, C₁₋₃ haloalkoxy, C₁₋₃ alkylcarbonyl, haloalkyl, C₁₋₄ hydroxyalkyl, and haloalkylcarbonyl. Another embodiment of the invention is a compound of Formula II or a pharmaceutically acceptable salt thereof, wherein R¹ is: i) substituted pyridyl or thienyl; or ii) optionally substituted furanyl, oxazolyl or thienyl, thiazolyl and the remainder of the variables are as described for Formula (II) Examples of substituents on R¹ include halo, C₁₋₄ alkyl, —OH, C₁₋₃ alkoxy, C₁₋₃ haloalkoxy, C₁₋₃ alkylcarbonyl, haloalkyl, C₁₋₄ hydroxyalkyl, and haloalkylcarbonyl (e.g., C₁₋₃ alkyl and C₁₋₃ hydroxyalkyl). In another embodiment, R¹ is represented by any one of Formulas (a)-(k), and the remainder of the variables are as described for Formula II.

Alternatively, R¹ is an optionally substituted 5-membered heteroaryl ring, wherein the remainder of the variables are as described for Formula II. In another aspect, R¹ is an optionally substituted furanyl, oxazolyl, thienyl, thiazolyl, pyrrolyl, or pyrazolyl, wherein the remainder of the variables are as described for Formula II. Examples of substituents on R¹ include C₁₋₄ alkyl, —C₁₋₃ alkoxy, C₁₋₃ haloalkoxy, C₁₋₃ alkylcarbonyl, haloalkyl, C₁₋₄ hydroxyalkyl, and haloalkylcarbonyl.

In a third compound embodiment, the invention relates to compounds of Formula II or a pharmaceutically acceptable salt thereof, wherein R¹ is as described in the second compound embodiment; R² is —NH(CH₂)ₙCOR⁴ or —NH(CH₂)ₙSO₂R⁴; R³ is H; R⁴ is phenyl, naphthyl, quinolinyl, pyridyl, furanyl, oxazolyl, thiazolyl, imidazolyl, thienyl or indolyl, each optionally substituted by one or two R⁵; and each R⁵ is independently selected from F, Br, Cl, methyl, ethyl, isopropyl, S-methyl, S-ethyl, S-isopropyl, nitro, methoxy, ethoxy, propoxy and butoxy. Alternatively, R² is —H; R³ is NH(CH₂)ₙCOR⁴ or —NH(CH₂)ₙSO₂R⁴; R⁴ is phenyl, naphthyl, quinolinyl, pyridyl, furanyl or indolyl, each optionally substituted by one or two R⁵; and each R⁵ is independently selected from F, Br, Cl, methyl, ethyl, propyl, S-methyl, S-ethyl, S-isopropyl, nitro, methoxy, ethoxy, propyl and butoxy.

In a fourth compound embodiment, the invention relates to compounds of Formula II or a pharmaceutically acceptable salt thereof, wherein R¹ is as described in the second compound embodiment; R² is hydrogen; R³ is —NHCOR⁴ or —NHSO₂R⁴; and R⁴ is a phenyl or pyridyl, each optionally substituted with one or two R⁵, wherein R⁵ is as described for Formula II. Alternatively, R² is —NHCOR⁴ or —NHSO₂R⁴; R³ is hydrogen; and R⁴ is an phenyl or pyridyl, each optionally substituted with one or two R⁵ wherein R⁵ is as described for Formula II. In one aspect, R⁴ is phenyl substituted by one or two R⁵ independently selected from fluoro, chloro, bromo, —S(methyl), —S(ethyl), —S(isopropyl), methyl, ethyl, trifluoromethyl, methoxy and ethoxy, or R⁴ is a pyridyl optionally substituted substituted by one R⁵ selected from fluoro, chloro, —S(isopropyl), methyl, methoxy or ethoxy.

The compounds of the invention are useful for treating cancers that show a dependence on Rae protein signaling for their growth and survival and, in particular, where the tumor progression is driven by dysregulation of Rac signaling. Specific examples include breast cancer, melanoma, ovarian cancer, head cancer, neck cancer, prostate cancer, colorectal cancer, pancreatic cancer, liver cancer, bladder cancer, non-Hodgkin's lymphoma, and leukemia (acute lymphoblastic leukemia, chronic myeloid leukemia, acute myeloid leukemia). Additionally, the compounds of the invention can be used to treat kidney disease and heart disease.

The invention also provides a method to treat a condition characterized by excessive or undesired levels of activity of Rac1, wherein the method comprises administering to a subject in need of such treatment an effective amount of a compound of Formula (I) or any subgenus thereof as described herein, or a pharmaceutical composition comprising such compound. The subject can be a mammal, and is preferably a human, and is typically a subject diagnosed with a condition associated with excessive activity of Rac1. Conditions treatable by the compounds and methods described herein include various forms of cancer that are responsive to Rac1 inhibitors, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as AML or CML, multiple myeloma, lymphoid disorders, skin cancers including melanoma, neuroblastoma, ovarian cancer, pancreatic cancer, prostate cancer, rectal cancer, sarcoma, testicular cancer, and thyroid cancer.

The compounds are especially indicated for use to treat melanoma, ovarian cancer, thyroid cancer, colon cancer, breast cancer, and prostate cancer. Indications of special interest for use of the compounds of the invention include cancers that express Rac1b, a splice variant of Rac1, and the mutant forms Rac1 P29S and Rac1 A149V. Examples of breast cancer for use of the compounds include ER+ breast cancer that is resistant to ER targeted therapy and/or CDK 4/6 inhibitors; and HER2+ breast cancer that is resistant to HER2 targeted therapies. Examples of prostate cancer for use of the compounds include castrate resistant prostate cancer (CRPC) that resistant to androgen receptor inhibitors. Examples of ovarian cancer for use of the compounds include ovarian cancer that is platinum resistant and for patients who are HRD negative.

In one aspect, the cancer is determined to exhibit a high expression level of Rac1 prior to treatment with a therapeutically effective amount of a Rac1 inhibitor. This determination can be made by routine diagnostic methods which obtain cancer cells from a patient. These methods include, but are not limited to, biopsy, blood tests, and other diagnostic methods which obtain samples of cancer cells such as tissue samples, circulating tumor cells, exosomes, or biomolecules characteristic of cancer such as circulating nucleic acids or proteins. The expression level of Rac1 in the cancer cells is then determined or inferred. Determining if the cancer exhibits a high expression level of Rac1 is by methodology known in the art, for example, by determining Rac1 expression levels in the isolated cancer cells by RNA sequencing (RNA-Seq), microarray, quantitative PCR, or NanoString™ gene expression panels, gene amplification by FISH, or Rac1 protein by immunohistochemistry, flow cytometry, immunocytochemistry or Western blot. See e.g., RT-qPCR analysis discussed below. In one embodiment, the methods disclosed herein further comprise a step of performing a biopsy of the patient's cancer prior to treatment and determining from the cancer cells isolated from the biopsy if the cancer (cancer cells) exhibits a high expression level of Rac1.

In one embodiment, the Rac1 expression level is determined for Rac1 wild type (Rac1 wt). In another embodiment, the Rac1 expression level is determined for a genomic variant of Rac1, such as the Rac1b splice variant and the Rac1 P29S mutant.

In another aspect, the invention is a method of treating a patient with a cancer comprising providing cancer cells from the cancer patient; determining the expression level of Rac1 in the cancer cells; and administering to the patient a therapeutically effective amount of a Rac1 inhibitor, if the patient's cancer (cancer cells) exhibits a high expression level of Rac1 In one embodiment, the method further comprises excluding the patient from administration of a Rac1 inhibitor if the patient's cancer (cancer cells) does not exhibit a high expression level of Rac1. The cancer cells used in the present invention can be obtained from a sample which is, but not limited to a sample of tissue, blood (including blood fractions), lymphatic fluid, sputum, feces, urine, bronchial lavage, or other body fluid.

In another aspect, provided herein is a method of selecting a patient who is likely to respond to treatment with a Rac1 inhibitor, said method comprising determining the expression level of Rac1 of a cancer of the patient, wherein the patient is likely to respond to treatment if the expression level of Rac1 by the cancer is high.

In another aspect, provided herein is a method of treating a patient with a cancer, comprising determining the expression level of Rac1 of the cancer and administering a therapeutically effective amount of a Rac1 inhibitor if the expression level of Rac1 by the cancer is high, and treating the patient with an anti-cancer therapy other than a Rac1 inhibitor if the patient's cancer does not exhibit a high expression level of Rac1.

In one aspect, the high expression level of Rac1 is characterized by an expression level falling within the top 50% of Rac1 expression levels of the cancer cells from the same cancer type in a random population of patients. The Rac1 expression level can be obtained from methods suitable for determining Rac1 expression levels, such as, e.g., expression levels derived from RNA-sequencing such as normalized read counts and TPM (Transcripts Per Million) or normalized cycle threshold (Ct) levels from RT-PCR measurements] for Rac1 robustly standardized (quantiles 2.5% and 97.5% set to −1 and +1, respectively).

As used herein “high expression” means an expression level falling within the top 5%, 10%, 15%, 20%, 25%, 30%, 40%, 45%, or 50% of the expression levels. “Top 50%”, for example, can be obtained by collecting expression levels of Rac1 from the cancer cells (e.g., from tissue samples) of a random population of subjects, e.g., at least 25 subjects, at least 50 subjects, at least 100 subjects, at least 500 subjects, at least 1000 subjects or the like, having the same cancers and then assessing whether the expression level of a new subject falls within the top 50% percentile.

In an alternative, “high expression” refers to a level of Rac1 in the cancer from the patient above a defined reference level of 25%, 30%, 40%, 50%, 60%, 70%, 80%, 85%, 90%, 95%, 100%, 150%, 200%, 250% or greater, determined by the methods described herein, as compared to the reference level.

“Reference level” refers to an average Rac1 expression level determined in cells of the same cell type as the cancer obtained from a population of healthy individuals without the cancer. In an alternative aspect, the reference level can be determined in non-cancerous cells of the same cell type as the cancer obtained from the patient.

In one aspect, the reference level can be obtained by determining the average normalized Rac1 expression [which can be obtained from methods suitable for determining Rac1 expression levels, such as, e.g., expression levels derived from RNA-sequencing such as normalized read counts and TPM (Transcripts Per Million) or normalized cycle threshold (Ct) levels from RT-PCR measurements] for Rac1 robustly standardized (quantiles 2.5% and 97.5% set to −1 and +1, respectively).

Some genomic variants of Rac1, such as Rac1 P29S, are only known to be expressed in cancerous cells. Thus, for such genomic variants, “high Rac1 expression” means a detectable level of such Rac1 genomic variant. Rac1 P29S is most prevalent in melanoma. Accordingly, one embodiment of this invention relates to a method of treating a cancer that expresses Rac P29S in a patient by administering to the patient an effective amount of a Rac1 P29S inhibitor of this invention. In one aspect, the cancer that expresses Rac1 P29S is melanoma.

Another embodiment of this invention provides a method of treating a cancer that is characterized by high expression of Rac1 b in a patient by administering to the patient an effective amount of a Rac1b inhibitor. In one aspect, the cancer that highly expresses Rac1b is colorectal, non-small cell lung, small cell lung, breast, prostate, thyroid, hepatocellular, ovarian, esophageal, gastric, or pancreatic cancer.

Elevated levels of activated Rac1 or GTP-bound Rac1 are found in patients that have high expression levels of certain other proteins that activate Rac1 such as guanine nucleotide exchange factors (GEFs). At least 20 GEFs are involved in Rac1 activation. The GEFs Tiam1 and P-Rex1 are Rac1 specific. Accordingly, one embodiment of this invention relates to treating a cancer that is characterized by high expression levels of a GEF in a patient by administering to the patient an effective amount of a Rac1 inhibitor. In one aspect, the cancer is characterized by high expression levels of Tiam1 or P-Rex1. Methods for determining the expression levels of the GEF are available to those skilled in the art in manners that are analogous to those described above for determining the expression levels of Rac1.

Elevated levels of activated Rac1 or GTP-bound Rac1 are also found in patients that have low expression levels of proteins that are involved in the degradation of active Rac1. HACE-1 is an E3-ubiquitin ligase tumor suppressor that targets active Rac1 for degradation. Low levels of HACE-1 are associated with higher levels of active, GTP-bound Rac1. Accordingly, one embodiment of this invention relates to treating a cancer that is characterized by low expression levels of HACE-1 in a patient by administering to the patient an effective amount of a Rac1 inhibitor. Methods for determining the expression levels of HACE-1 are available to those skilled in the art in manners that are analogous to those described above for determining the expression levels of Rael.

In one aspect, the low expression level of HACE-1 is characterized by an expression level falling within the bottom 50% of HACE-1 expression levels of the cancer cells from the same cancer type in a random population of patients. The HACE-1 expression level can be obtained from methods suitable for determining HACE-1 expression levels, such as, e.g., expression levels derived from RNA-sequencing such as normalized read counts and TPM (Transcripts Per Million) or normalized cycle threshold (Ct) levels from RT-PCR measurements] for Rac1 robustly standardized (quantiles 2.5% and 97.5% set to −1 and +1, respectively).

As used herein “low expression” means an expression level falling within the bottom 5%, 10%, 15%, 20%, 25%, 30%, 40%, 45%, or 50% of the expression levels. “Bottom 50%”, for example, can be obtained by collecting expression levels of HACE-1 from the cancer cells (e.g., from tissue samples) of a random population of subjects, e.g., at least 25 subjects, at least 50 subjects, at least 100 subjects, at least 500 subjects, at least 1000 subjects or the like, having the same cancers and then assessing whether the expression level of a new subject falls within the bottom 50% percentile.

In an alternative, “low expression” refers to a level of HACE-1 in the cancer from the patient below a defined reference level by 25%, 30%, 40%, 50%, 60%, 70%, 80%, 85%, 90%, 95%, 100%, 150%, 200%, 250% or greater, determined by the methods described herein, as compared to the reference level.

Levels of HACE-1 may also be determined using known immunohistochemistry methods for the HACE-1 protein. See Da et al., Signal Transduction and Targeted Therapy 6, Article Number 399 (2021) and Anglesio et al., Human Molecular Genetics 13(18), pp 2061-2074 (2004).

The invention is illustrated by the following examples, which are not intended to be limiting in any way.

### EXEMPLIFICATION

### Abbreviations Used in Experimental Procedures:

DMF Dimethylformamide
EtOH Ethanol
TEA Triethylamine
DIPEA N,N-Diisopropylethylamine
dppf 1,1′-Bis(diphenylphosphino)ferrocene
aq. Aqueous
EtOAc Ethyl acetate
MeOH Methanol
RT Room temperature
TLC Thin layer chromatography
DCM Dichloromethane
THF Tetrahydrofuran
RT Room temperature
tBuOH tert-butanol
MeCN Acetonitrile

### Synthesis of Common Intermediate 11

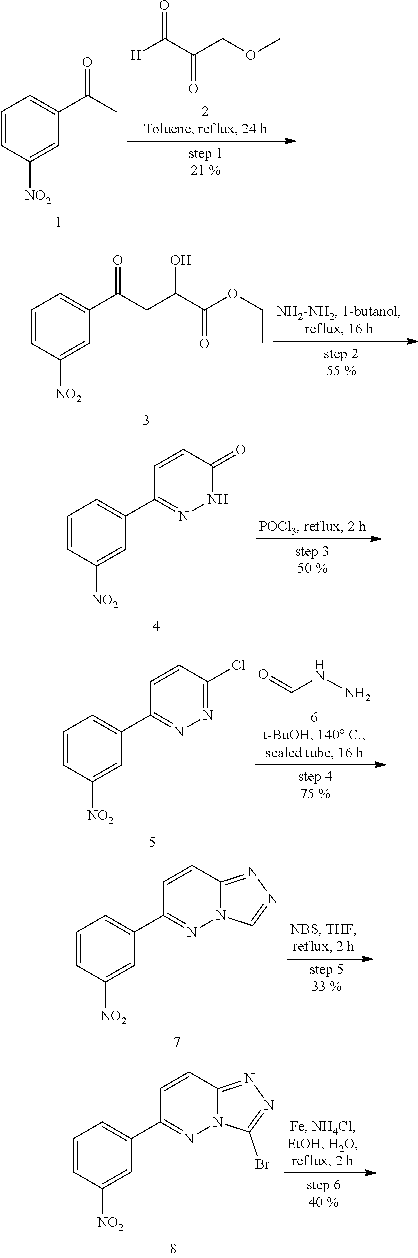

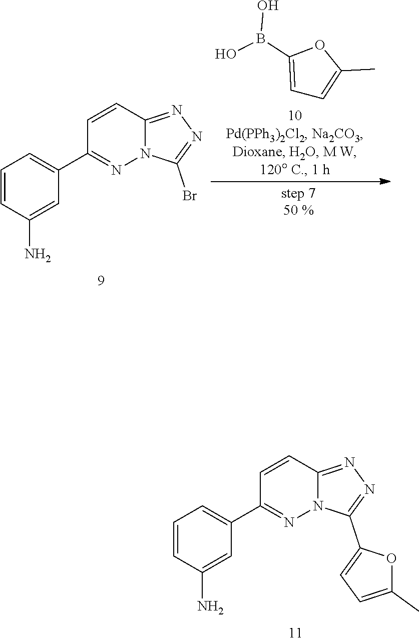

### Step 1:

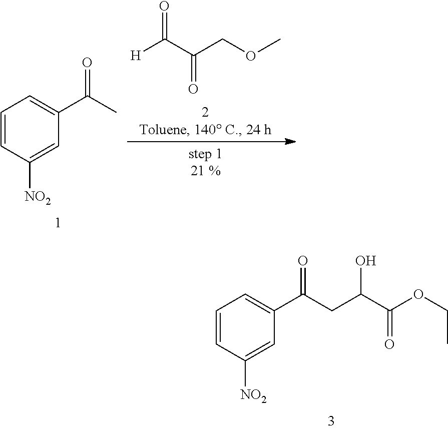

Ethyl 2-hydroxy-4-(3-nitrophenyl)-4-oxobutanoate (3): A mixture of compound 1 (50 g, 0.30 mol) and 50% ethyl glyoxylate (62 ml, 0.30 mol) in toluene (100 ml) was heated to 140° C. for 24 h. The progress of the reaction was monitored by TLC. After completion of the reaction, the reaction mixture was concentrated under reduced pressure and the obtained crude residue was purified by column chromatography (100-200 silica gel, 25-30% EtOAc-Hexane) to give 3 (17 g, yield: 21%) as an off-white solid (observed solid upon storing in the refrigerator over 6 h time).

¹H NMR [400 MHz, DMSO-d₆]: δ 8.35 (m, 2H), 8.2 (m, 2H), 4.52 (m, 1H), 4.1 (q, J=7.6 Hz, 2H), 3.45 (d, J=6.0 Hz, 2H), 1.7 (t, J=7.6 Hz, 3H).

LCMS: m/z: 266.17 [M−H]⁻, 92.50% (1.23 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

R_{f}=0.2 (Mobile phase: 50% EtOAc-Hexane).

### Step 2:

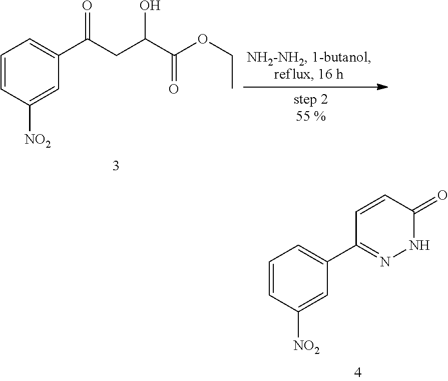 [SMILES: CCOC(=O)C(O)CC(=O)c1cccc([N+](=O)[O-])c1 ; O=c1ccc(-c2cccc([N+](=O)[O-])c2)n[nH]1]

6-(3-nitrophenyl)pyridazin-3(2H)-one (4): A mixture of compound 3 (17 g, 0.063 mol), hydrazine hydrate (2.5 ml, 0.076 mol) and 1-butanol (170 ml) was stirred at 120° C. for 16 h. The progress of the reaction was monitored by TLC. After completion of the reaction, the reaction mixture was cooled to room temperature, yellow solid was formed. The yellow solid was filtered, washed with 1-butanol, and dried to afford compound 4 (7.6 g, yield: 55%) which was used in the next step without any further purification.

¹H NMR (400 MHz, DMSO-d₆): δ 9.85 (s, 1H), 8.5 (d, J=9.2 Hz, 1H), 8.4 (m, 4H), 8.12 (d, J=5.2 Hz, 1H).

LCMS: m/z: 216.15 [M−1]⁻, 90.85% (1.12 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90),

Flow Rate: 0.4 mL/min.

R_{f}=0.4 (Mobile phase: 30% EtOAc-Hexane).

### Step 3:

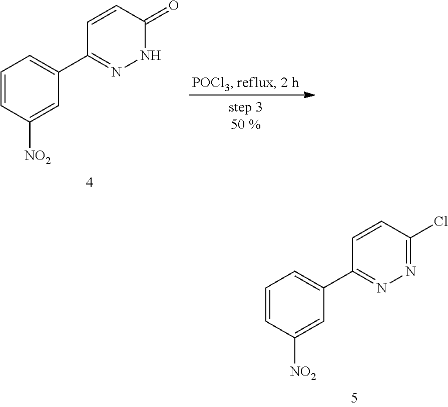 [SMILES: O=c1ccc(-c2cccc([N+](=O)[O-])c2)n[nH]1 ; O=[N+]([O-])c1cccc(-c2ccc(Cl)nn2)c1]

3-chloro-6-(3-nitrophenyl)pyridazine (5): A mixture of compound 4 (10 g, 0.046 mol) and POCl₃ (120 ml, 12 Vol) was refluxed for 2 h. The progress of the reaction was monitored by TLC. After completion of the reaction, the reaction mixture was diluted with ice-cold water and brown solid was formed. The solid was filtered and dried under reduced pressure to afford 5 (5.5 g, yield: 50%) which was used in the next step without any further purification.

¹H NMR (400 MHz, DMSO-d₆): δ 8.5 (d, J=9.2 Hz, 1H), 8.4 (m, 4H), 8.12 (d, J=5.2 Hz, 1H).

LCMS: m/z: 236.30 [M+1]⁺, 95.02% (1.39 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90),

Flow Rate: 0.4 mL/min.

R_{f}=0.6 (Mobile phase: 30% EtOAc-Hexane).

### Step 4:

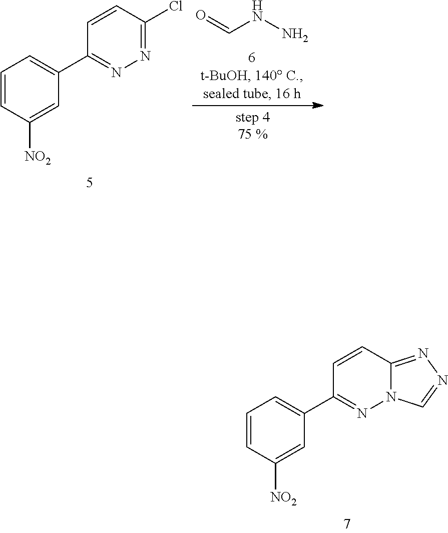

6-(3-nitrophenyl)-[1,2,4]triazolo[4,3-b]pyridazine (7): A mixture of compound 5 (5.0 g, 0.021 mol), formic-hydrazide (3.8 g, 0.063 mol) and 1-butanol (30 ml) in a sealed tube was stirred at 140° C. for 16 h. The progress of the reaction was monitored by TLC. After completion of the reaction, the reaction mixture was cooled to room temperature, and observed the formation of a yellow solid. The yellow solid was filtered and purified by silica gel column chromatography to afford compound 7 (3.85 g, yield: 75%).

¹H NMR (400 MHz, DMSO-d₆): δ 9.4 (s, 1H), 8.5 (d, J=9.2 Hz, 1H), 8.4 (m, 4H), 8.12 (d, J=5.2 Hz, 1H).

LCMS: m/z: 242.30 [M+1]⁺, 95.02% (1.10 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90),

Flow Rate: 0.4 mL/min.

R_{f}=0.4 (Mobile phase: 10% MeOH-DCM).

### Step 5:

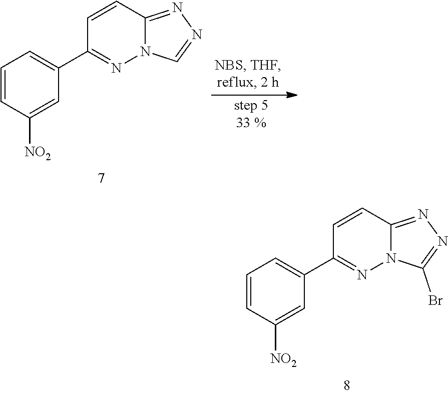 [SMILES: O=[N+]([O-])c1cccc(-c2ccc3nncn3n2)c1 ; O=[N+]([O-])c1cccc(-c2ccc3nnc(Br)n3n2)c1]

3-(3-bromo-[1,2,4]triazolo[4,3-b]pyridazin-6-yl)aniline (8): To a stirred solution of compound 7 (4.5 g, 0.018 mol) in THF (90.0 Vol) was added NBS (recrystallized from hot water) (4.0 g, 0.022 mol) in portion wise (each lot 1.0 eq up to 2.0 eq) at room temperature. The resultant reaction mixture was refluxed for 2 h. The progress of the reaction was monitored by TLC. After completion of the reaction, the reaction mixture was cooled to room temperature, quenched with NaHCO₃ solution, and extracted with ethyl acetate. The combined organic layer was dried over Na₂SO₄, filtered, and concentrated under reduced pressure to afford the crude residue which was purified by column chromatography (eluted in 1-2% MeOH-DCM) to give compound 8 (2 g, yield: 33%) as yellow color solid.

¹H NMR (400 MHz, DMSO-d₆): δ 8.9 (m, 1H), 8.6 (m, 2H), 8.47 (dd, J=1.6 Hz, 1H), 8.21 (d, J=10.0 Hz, 1H), 7.92 (m, 1H).

LCMS: m/z: 320.15 [M+1]+, 85.0% (1.26 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

R_{f}=0.5 (Mobile phase: 10% MeOH-DCM).

### Step 6:

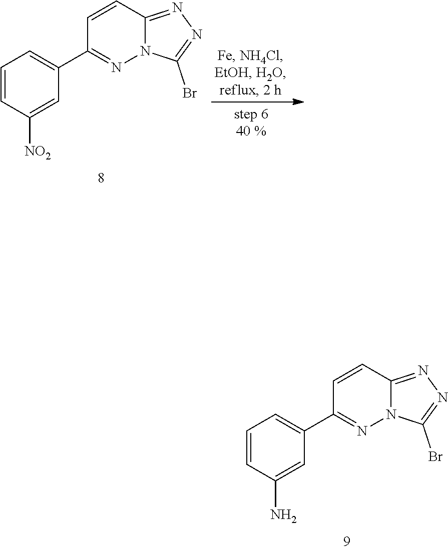 [SMILES: O=[N+]([O-])c1cccc(-c2ccc3nnc(Br)n3n2)c1 ; Nc1cccc(-c2ccc3nnc(Br)n3n2)c1]

3-(3-bromo-[1,2,4]triazolo[4,3-b]pyridazin-6-yl)aniline (9): To a stirred solution of compound 8 (1.8 g, 6.2 mmol) in ethanol:water (1:1) (50 ml, 20 Vol) was added NH₄Cl (3 g, 62 mmol) and iron powder (3 g, 62 mmol) respectively at room temperature. The resultant reaction mixture was refluxed for 2 h. The progress of the reaction was monitored by TLC. After completion of the reaction, the reaction mixture (hot condition) was filtered through a Celite bed and washed with ethanol. The filtrate was concentrated under reduced pressure. The residue obtained was diluted with water and extracted with DCM (3×50 mL). The combined organic extracts were dried over anhydrous Na₂SO₄, filtered, and concentrated under reduced pressure. The solid obtained was purified by column chromatography (eluted in 1-2% MeOH-DCM) to afford compound 9 (700 mg, yield: 40%) as yellow color solid.

¹H NMR (400 MHz, DMSO-d₆): δ 8.9 (m, 1H), 8.6 (m, 2H), 8.47 (dd, J=1.6 Hz, 1H), 8.21 (d, J=10.0 Hz, 1H), 7.92 (m, 1H), 4.2 (s, 2H).

LCMS: m/z: 290.15 [M+1]+, 85.0% (1.02 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

R_{f}=0.4 (Mobile phase: 5% MeOH-DCM).

### Step 7:

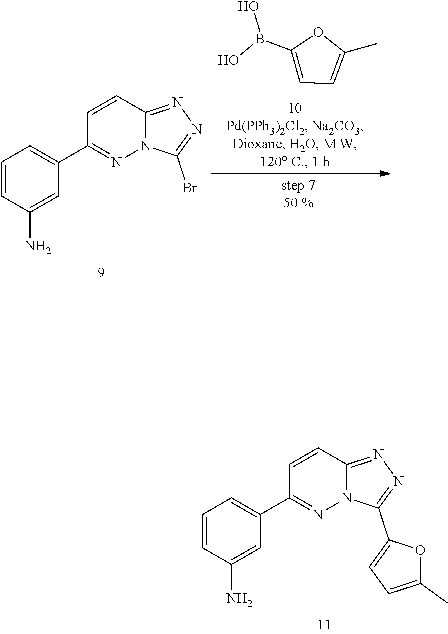

3-(3-(5-methylfuran-2-yl)-[1,2,4]triazolo[4,3-b]pyridazin-6-yl)aniline (11): To a stirred solution of compound 9 (300 mg, 1.0 mmol) in dioxane (2.0 ml) was added K₂CO₃ (500 mg, 3.0 mmol) and compound 10 (210 mg, 2.0 mol) at room temperature under argon atmosphere. The reaction mixture was degassed for 10 minutes. Pd(PPh₃)₂Cl₂ (16 mg, 0.01 mmol) was added and degassed the resultant reaction mixture for another 10 minutes. The reaction mixture was heated at 120° C. in a microwave reactor for 1 h. The progress of the reaction was monitored by TLC. After completion of the reaction, water was added to the reaction mixture and extracted with ethyl acetate. The combined organic layer was dried over Na₂SO₄, filtered and the filtrate was concentrated under reduced pressure to afford the crude. The crude residue obtained was purified by column chromatography (eluted in 2-3% MeOH-DCM) to afford compound 11 (150 mg, yield: 50%) as pale-yellow color solid.

¹H NMR [400 MHz, DMSO-d₆]: 6 8.9 (t, 1H), 8.6 (m, 2H), 8.47 (dd, J=1.6 Hz, 1H), 8.21 (d, J=10.0 Hz, 1H), 7.92 (t, 1H), 7.8 (d, J=7.2 Hz, 1H), 6.5 (d, J=2.4 Hz, 1H), 4.2 (s, 2H), 2.45 (s, 3H).

LCMS: m/z: 292.15 [M+1]+, 87.0% (1.02 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

R_{f}=0.3 (Mobile phase: 10% MeOH-DCM).

### Step 8:

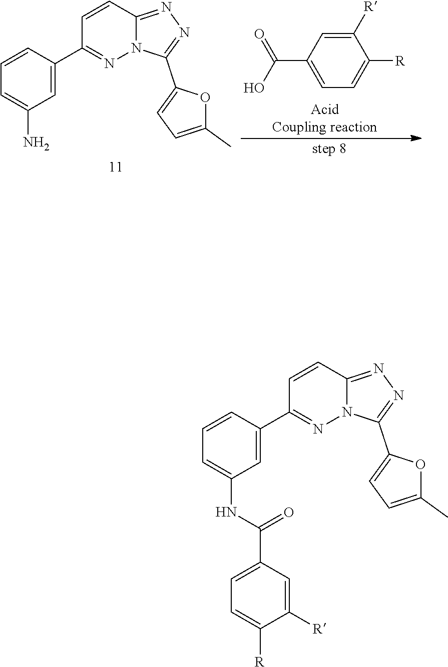

### General Procedure for Amide Couplings:

A: To a solution of acid (1.0 mol) in DMF (10.0 Vol) was added HATU (2.0 mol), DIPEA (3.0 mol), and compound 11 (1.0 mol) at room temperature and stirred for 16 hours. The reaction mixture was monitored by TLC. After completion of the reaction, the reaction mixture was diluted with ice-cold water, the solid was precipitated out, and the obtained solid was filtered and purified by C-18 reverse phase column chromatography by eluting 0-100% ACN-water to afford the desired product.

B: To a stirred solution of acid (1.0 mol) in Pyridine (5.0 Vol) was added T₃P (3.0 mol), followed by compound 11 (1.0 mol) at room temperature under argon atmosphere and stirred the reaction for 16 h at 90° C. The progress of the reaction was monitored by TLC. After completion of the reaction, the reaction mixture was diluted with ice-cold water, the solid was precipitated out, and the obtained solid was filtered and purified by C-18 reverse phase column chromatography by eluting 0-100% ACN-water to afford the desired product.

3-ethyl-N-(3-(3-(5-methylfuran-2-yl)-[1,2,4]triazolo[4,3-b]pyridazin-6-yl)phenyl)benzamide was prepared according to the general procedure B:

3-ethylbenzoic acid (62 mg) & 3-(3-(5-methylfuran-2-yl)-[1,2,4]triazolo[4,3-b]pyridazin-6-yl)aniline (11) (100 mg) to afford the title compound (25 mg, Yield: 14.4%).

LCMS (m/z)=424.23 [M+H]⁺ (LCMS purity 99.20%, 3.08 min).

1HNMR (400 MHz, DMSO-d6): δ (ppm) 10.5 (s, 1H), 8.69 (s, 1H), 8.56 (d, J=9.6 Hz, 1H), 8.04 (d, J=8.0 Hz, 1H), 7.96 (d, J=10.0 Hz, 1H), 7.92 (d, J=8.0 Hz, 1H), 7.8 (m, 2H), 7.6 (m, 2H), 7.5 (m, 2H), 6.5 (d, J=3.2 Hz, 1H), 2.71 (q, J=7.6 Hz, 2H), 2.47 (s, 3H), 1.26 (t, J=7.6 Hz, 3H).

3-ethyl-4-fluoro-N-(3-(3-(5-methylfuran-2-yl)-[1,2,4]triazolo[4,3-b]pyridazin-6-yl)phenyl)benzamide was prepared according to the general procedure A:

3-ethyl-4-fluorobenzoic acid (69 mg) & 3-(3-(5-methylfuran-2-yl)-[1,2,4]triazolo[4,3-b]pyridazin-6-yl)aniline (11) (120 mg) to afford the title compound (30 mg, Yield: 16.60%).

LCMS (m/z)=442.13 [M+H]⁺ (LCMS purity 98.83%, 3.12 min)

1HNMR (400 MHz, DMSO-d6) δ (ppm) 10.5 (s, 1H), 8.66 (s, 1H), 8.54 (d, J=10.0 Hz, 1H), 8.0-7.9 (m, 5H), 7.6 (m, 2H), 7.3 (m, 1H), 6.5 (d, J=3.2 Hz, 1H), 2.71 (q, J=7.6 Hz, 2H), 2.47 (s, 3H), 1.25 (t, J=7.6 Hz, 3H).

3-chloro-N-(3-(3-(5-methylfuran-2-yl)-[1,2,4]triazolo[4,3-b]pyridazin-6-yl)phenyl)benzamide was prepared according to the general procedure A:

3-chlorobenzoic acid (81 mg) & 3-(3-(5-methylfuran-2-yl)-[1,2,4]triazolo[4,3-b]pyridazin-6-yl)aniline (11) (150 mg) to afford the title compound (30 mg, Yield: 13.6%).

LCMS (m/z)=430.11 [M+H]⁺ (LCMS purity 95.64%, 6.32 min)

¹HNMR (400 MHz, DMSO-d6): δ (ppm): 10.61 (s, 1H), 8.67 (s, 1H), 8.55 (d, J=9.6 Hz, 1H), 7.96 (m, 5H), 7.62 (m, 4H), 6.5 (s, 1H), 2.45 (s, 3H).

3-chloro-4-fluoro-N-(3-(3-(5-methylfuran-2-yl)-[1,2,4]triazolo[4,3-b]pyridazin-6-yl)phenyl)benzamide was prepared according to the general procedure A:

3-chloro-4-fluorobenzoic acid (90 mg) & 3-(3-(5-methylfuran-2-yl)-[1,2,4]triazolo[4,3-b]pyridazin-6-yl)aniline (11) (150 mg) to afford the title compound (30 mg, Yield: 13.0%).

LCMS (m/z)=448.17 [M+H]⁺ (LCMS purity 99.1%, 3.08 min)

¹HNMR (400 MHz, DMSO-d6): δ (ppm): 10.63 (s, 1H), 8.66 (s, 1H), 8.53 (d, J=10.4 Hz, 1H), 8.24 (m, 1H), 8.03 (m, 4H), 7.60 (m, 3H), 6.51 (d, J=2.4 Hz, 1H), 2.47 (s, 3H).

4-fluoro-N-(3-(3-(5-methylfuran-2-yl)-[1,2,4]triazolo[4,3-b]pyridazin-6-yl)phenyl)benzamide was prepared according to the general procedure A:

4-fluorobenzoic acid (72 mg) & 3-(3-(5-methylfuran-2-yl)-[1,2,4]triazolo[4,3-b]pyridazin-6-yl)aniline (11) (150 mg) to afford the title compound (60 mg, Yield: 28.16%).

LCMS (m/z)=414.46 [M+H]⁺ (LCMS purity 95.73%, 2.82 min)

¹HNMR (400 MHz, DMSO-d6): δ (ppm): 10.57 (s, 1H), 8.67 (s, 1H), 8.55 (d, 1H, J=10.0 Hz), 8.11 (m, 2H), 8.0 (m, 3H), 7.61 (m, 2H), 7.41 (m, 2H), 6.50 (d, 1H, J=2.8 Hz), 2.47 (s, 3H).

3-(isopropylthio)-N-(3-(3-(5-methylfuran-2-yl)-[1,2,4]triazolo[4,3-b]pyridazin-6-yl)phenyl)benzamide was prepared according to the general procedure A:

3-(isopropylthio)benzoic acid (67 mg) & 3-(3-(5-methylfuran-2-yl)-[1,2,4]triazolo[4,3-b]pyridazin-6-yl)aniline (11) (100 mg) to afford the title compound (25 mg, Yield: 15.5%).

LCMS (m/z) 6470.18 [M+H]⁺ (LCMS purity 98.69%, 3.24 mi)

¹HNMR (400 MHz, DMSO-d6) δ (ppm): 10.57 (s, 1H), 8.67 (s, 1H), 8.56 (d, J=10.0 Hz, 1H), 8.0-7.86 (m, 4H), 7.8 (d, J=7.2 Hz, 1H), 7.6 (m, 4H), 6.5 (d, J=2.4 Hz, 1H), 3.6 (m, 1H), 2.5 (s, 3H), 1.29 (d, J=6.0 Hz, 6H).

TABLE 1
Analytical data

| S. | Substrate | Substrate | | | Temp/ | | |
|---|---|---|---|---|---|---|---|
| No | 1 | 2 | Product | Condition | Time | Yield | LCMS & 1H NMR |
| 1 | 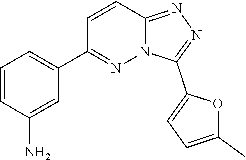 | 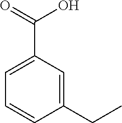 | 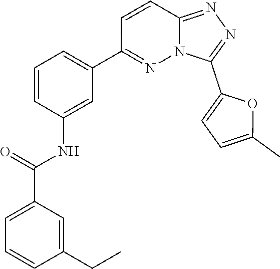 [SMILES: CCc1cccc(C(=O)Nc2cccc(-c3ccc4nnc(-c5ccc(C)o5)n4n3)c2)c1] | T₃P, Pyridine, 90° C., 16 h | 90° C./ 16 h | 14.4% | LCMS (m/z) = 424.23 [M + H]⁺ (LCMS purity 99.20%, 3.08 min) 1HNMR (400 MHz, DMSO-d6) δ(ppm) 10.5 (s, 1H), 8.69 (s, 1H), 8.56 (d, J = 9.6 Hz, 1H), 8.04 (d, J = 8.0 Hz, 1H), 7.96 (d, J = 10.0 Hz, 1H), 7.92 (d, J = 8.0 Hz, 1H), 7.8 (m, 2H), 7.6 (m, 2H), 7.5 (m, 2H), 6.5 (d, J = 3.2 Hz, 1H), 2.71 (q, J = 7.6 Hz, 2H), 2.47 (s, 3H), 1.26 (t, J = 7.6 Hz, 3H). |
| 2 | 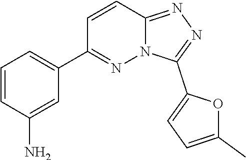 | 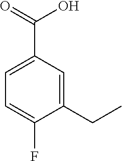 | 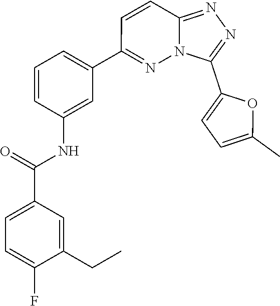 [SMILES: CCc1cc(C(=O)Nc2cccc(-c3ccc4nnc(-c5ccc(C)o5)n4n3)c2)ccc1F] | HATU, DIPEA, DMF, RT, 16 h | 25° C./ 16 h | 16.6% | LCMS (m/z) = 442.13 [M + H]⁺ (LCMS purity 98.83%, 3.12 min) 1HNMR (400 MHz, DMSO-d6) δ(ppm) 10.5 (s, 1H), 8.66 (s, 1H), 8.54 (d, J = 10.0 Hz, 1H), 8.0-7.9 (m, 5H), 7.6 (m, 2H), 7.3 (m, 1H), 6.5 (d, J = 3.2 Hz, 1H), 2.71 (q, J = 7.6 Hz, 2H), 2.47 (s, 3H), 1.25 (t, J = 7.6 Hz, 3H). |
| 3 | 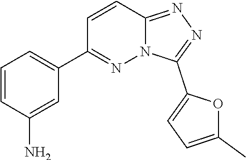 | 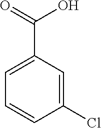 | 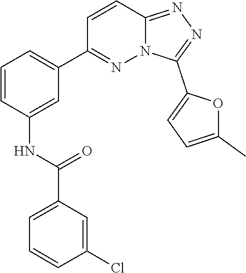 [SMILES: Cc1ccc(-c2nnc3ccc(-c4cccc(NC(=O)c5cccc(Cl)c5)c4)nn23)o1] | HATU, DIPEA, DMF, RT, 16 h | 25° C./ 16 h | 13.6% | LCMS (m/z) = 430.11 [M + H]⁺ (LCMS purity 95.64%, 6.32 min) ¹HNMR (400 MHz, DMSO-d6): δ (ppm): 10.61 (s, 1H), 8.67 (s, 1H), 8.55 (d, J = 9.6 Hz, 1H), 7.96 (m, 5H), 7.62 (m, 4H), 6.5 (s, 1H), 2.45 (s, 3H). |
| 4 | 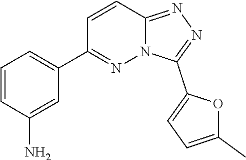 | 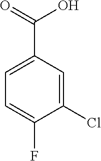 [SMILES: O=C(O)c1ccc(F)c(Cl)c1] | 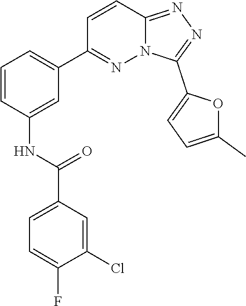 [SMILES: Cc1ccc(-c2nnc3ccc(-c4cccc(NC(=O)c5ccc(F)c(Cl)c5)c4)nn23)o1] | HATU, DIPEA, DMF, RT, 16 h | 25° C./ 16 h | 13.0% | LCMS (m/z) = 448.17 [M + H]⁺ (LCMS purity 99.1%, 3.08 min) ¹HNMR (400 MHz, DMSO-d6): □ (ppm): 10.63 (s, 1H), 8.66 (s, 1H), 8.53 (d, J = 10.4 Hz, 1H), 8.24 (m, 1H), 8.03 (m, 4H), 7.60 (m, 3H), 6.51 (d, J = 2.4 Hz, 1H), 2.47 (s, 3H). |
| 5 | 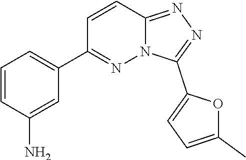 | 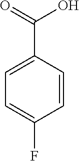 | 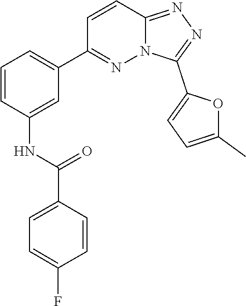 [SMILES: Cc1ccc(-c2nnc3ccc(-c4cccc(NC(=O)c5ccc(F)cc5)c4)nn23)o1] | HATU, DIPEA, DMF, RT, 16 h | 25° C./ 16 h | 28.1% | LCMS (m/z) = 414.46 [M + H]⁺ (LCMS purity 95.73%, 2.82 min) ¹HNMR (400 MHz, DMSO-d6): (ppm): 10.57 (s, 1H), 8.67 (s, 1H), 8.55 (d, J = 10.0 Hz, 1H), 8.11 (m, 2H), 8.0 (m, 3H), 7.61 (m, 2H), 7.41 (m, 2H), 6.50 (d, J = 2.8 Hz, 1H), 2.47 (s, 3H). |

| | | | | | | | |
|---|---|---|---|---|---|---|---|
| 6 | 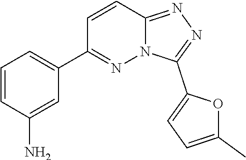 | 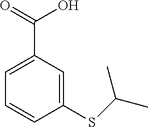 | 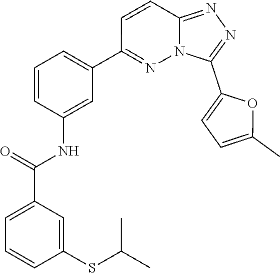 [SMILES: Cc1ccc(-c2nnc3ccc(-c4cccc(NC(=O)c5cccc(SC(C)C)c5)c4)nn23)o1] | HATU, DIPEA, DMF, RT, 16 h | 25° C./ 16 h | 15.5% | LCMS (m/z) = 470.18 [M + H]⁺ (LCMS purity 98.69%, 3.24 min) 1HNMR (400 MHz, DMSO-d6) δ(ppm) 10.57 (s, 1H), 8.67 (s, 1H), 8.56 (d, J = 10.0 Hz, 1H), 8.0-7.86 (m, 4H), 7.8 (d, J = 7.2 Hz, 1H), 7.6 (m, 4H), 6.5 (d, J = 2.4 Hz, 1H), 3.6 (m, 1H), 2.5 (s, 3H), 1.29 (d, J = 6.0 Hz, 6H). |

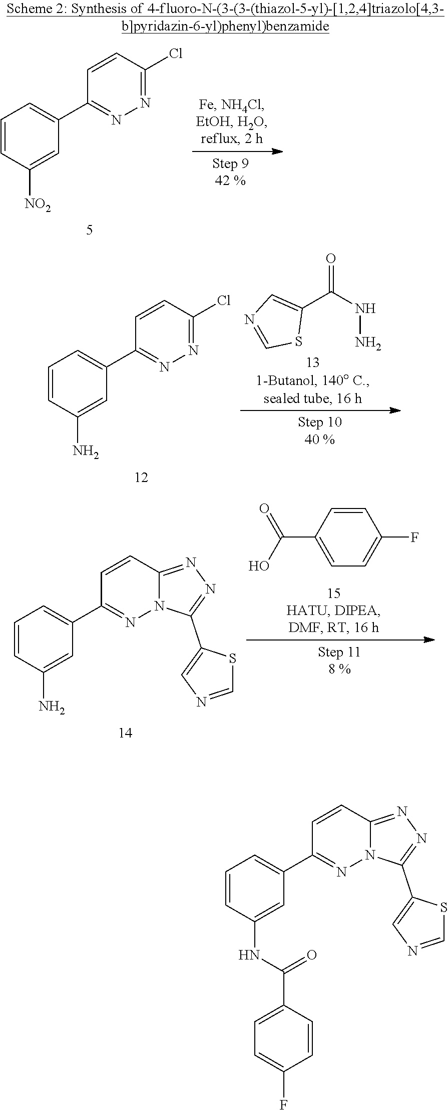

### Step-9:

3-(6-chloropyridazin-3-yl) aniline (12): To a stirred solution of compound 5 (0.5 g, 2.21 mmol) in ethanol:water (1:1) (12 ml, 20 Vol) was added NH₄Cl (1.14 g, 21.22 mmol) and iron powder (1.19 g, 21.22 mmol) at room temperature. The resultant reaction mixture was refluxed for 2 h. The progress of the reaction was monitored by TLC. After completion of the reaction, the reaction mixture (hot condition) was filtered through a Celite bed and washed with ethanol. The filtrate was concentrated under reduced pressure. The residue obtained was diluted with water and extracted with DCM (3×30 mL). The combined organic extracts were dried over anhydrous Na₂SO₄, filtered and the filtrate was concentrated under reduced pressure. The solid was purified by column chromatography (eluted in 1-2% MeOH-DCM) to afford compound 12 (185 mg, yield: 42%) as yellow color solid.

¹H NMR (400 MHz, DMSO-d₆): δ 8.34 (s, 1H), 8.23 (d, J=8.8 Hz, 1H), 8.0 (d, J=9.2 Hz, 1H), 7.69 (d, J=8.0 Hz, 1H), 7.6 (d, J=8.0 Hz, 1H), 7.44 (t, 1H), 4.2 (s, 2H).

LCMS: m/z: 206.15 [M+1]+, 95.64% (1.06 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

### Step-10:

3-(3-(thiazol-5yl)-[1,2,4]triazolo[4,3-b]pyridazin-6-yl)aniline (14): A mixture of compound 12 (0.870 mmol), hydrazine derivative 13 (2.63 mmol) and 1-butanol (10.0 Vol) in a sealed tube was stirred at 140° C. for 16 h. The progress of the reaction was monitored by TLC. After completion of the reaction, the reaction mixture was cooled to room temperature and observed precipitation. The precipitate was filtered, washed with 1-butanol, and used in the next step without any further purification

LCMS: m/z: 295.15 [M+1]+, 43.57% (1.06 min),

Column: Kinetex EVO C18 (2.1×50 mm, 1.7 ìm),

Mobile Phase: A—0.01% FA in water; B—0.01% FA in ACN,

(T/% B: 0.01/10, 0.2/10, 1.5/90, 3/90)

Flow Rate: 0.4 mL/min.

### Step-11:

4-fluoro-N-(3-(3-(thiazol-5-yl)-[1,2,4]triazolo[4,3-b]pyridazin-6-yl)phenyl)benzamide was prepared according to the general procedure (A):

Acid (48 mg) & Compound 14 (100 mg) to afford the title compound (11.12 mg, Yield: 08%).

LCMS (m/z)=417.16 [M+H]⁺ (LCMS purity 98.76%, 2.51 min)

¹HNMR (400 MHz, DMSO-d6) δ (ppm): 10.56 (s, 1H), 9.35 (s, 1H), 9.01 (s, 1H), 8.63 (s, 1H), 8.56 (d, J=10 Hz, 1H), 8.0 (m, 5H), 7.59 (t, J=8.0 Hz, 1H), 7.37 (t, J=8.8 Hz, 2H).

TABLE 2
Analytical data

| S. | Substrate | Substrate | | Condi- | Temp/ | | |
|---|---|---|---|---|---|---|---|
| No | 1 | 2 | Product | tion | Time | Yield | LCMS & 1H NMR |
| 1 | 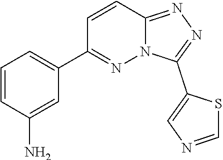 [SMILES: Nc1cccc(-c2ccc3nnc(-c4cncs4)n3n2)c1] | 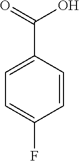 | 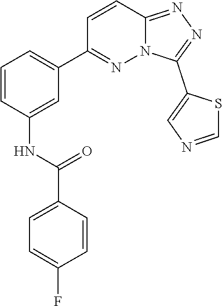 [SMILES: O=C(Nc1cccc(-c2ccc3nnc(-c4cncs4)n3n2)c1)c1ccc(F)cc1] | | 25° C./ 16 h | 8% | LCMS (m/z) = 417.16 [M + H]⁺ (LCMS purity 98.76%, 2.51 min) ¹HNMR (400 MHz, DMSO- d6): δ (ppm): 10.56 (s, 1H), 9.35 (s, 1H), 9.01 (s, 1H), 8.63 (s, 1H), 8.55 (d, J = 10 Hz, 1H), 8.0 (m, 5H), 7.59 (t, J = 8.0 Hz, 1H), 7.37 (t, J = 8.8 Hz, 2H). |

### Pharmacology

### In Vitro Assays

### 1. Cell Viability Assay (MCF7)

In order to determine a compound's effect on cell viability, PrestoBlue assays were performed as previously described by Kuhn et al. (2013) with modifications. MCF-7 human breast cancer and IGR-1 human melanoma cell lines were seeded into 96-well plates 24 hours prior to addition of drug. Cells are treated with 0 to 200 μM (concentrations) of compound solubilized n DMSO, adjusting the final concentration of DMSO to 1% in the well. Three days after drug treatment, cell viability was measured by adding PrestoBlue (Life Technologies Ltd.; cat. No. A13262) reagent directly to wells containing media/drug to reach a final concentration of 10%. PrestoBlue is a modified molecule of the common Alamar Blue probe used to determine viability based on the ability of a cell to metabolize a nonfluorescent compound (resazurin) to a florescent molecule (resorufin). Following a 1-hour incubation period, total well fluorescence was measured using the microplate reader M1000 pro (Tecan) with excitation 560-5 nm and emission 590-5 nm. Data is analyzed using the GraphPad Prism software (GraphPad Software, Inc.), and IC50 (dose leading to 50% cell death) was calculated from the dose-response curves. The percentage of living cells was then computed by comparison with control wells. Kuhn, Jonas et al., Assay and Drug Development Technologies, March 2013, Label-Free Cytotoxicity Screening Assay by Digital Holographic Microscopy.

### 2. Rac Activation AlphaScreen Assay (Rac1 AS)

AlphaScreen® assays were performed in 96-well microplates in a final reaction volume of 60 μL. Recombinant His-Rac1, recombinant GST-PBD (PAK Binding Domain), donor and acceptor beads (PerkinElmer), and inhibitors were incubated in exchange buffer (20 mM Tris pH 7.5, 50 mM NaCl, 1 mM MgCl₂, 1 mM EDTA, 500 nM GTPyS (guanosine 5′-[Y-thio]triphosphate)) at 37° C. Readings were performed on a Tecan M1000 pro microplate reader after 1 hour. Data was analyzed using the GraphPad Prism software (GraphPad Software, Inc.), and IC₅₀ (dose leading to 50% disruption of complex) was calculated from the dose-response curves.

### 3. Cell Migration Assay

An essential characteristic of malignant cells is their ability to migrate, invade host tissues and to produce metastases. In order to evaluate the capacity of one compound to affect the ability of tumoral cells to migrate, migration assays are performed using HUVEC cells. HUVECs (2.5×10 4) are seeded onto uncoated filters in a 24-well transwell Boyden chamber (8-mm pore size; Costar) and allowed to migrate in the presence and absence of different doses of the Rac1 inhibitor test material (5, 6.25, 10, 12.5, 20, 25 μM). The cells that migrated to the underside of the filter are stained with crystal violet and counted under the bright field microscopy.

### 4. Cellular Proliferation Assay

This assay measures the ability of the test material to inhibit the proliferation of HUVEC cells. 25,000 cells were seeded in 6-well plates. The next day, vehicle (DMSO) or test material were added to cells (Day 0). Cells were counted on days 0 through day 4 using a hemocytometer.

### 5. Western Blot Analysis

Western blot analysis is used to identify specific proteins from a complex mixture of proteins extracted from cells. Equal amount of protein is run on the SDS-PAGE gel and after separating the protein mixture, it is transferred to a membrane. The transferred protein is then probed with a combination of antibodies: one antibody specific to the protein of interest (primary antibody) and another antibody specific to the host species of the primary antibody (secondary antibody). The secondary antibody is complexed with an enzyme, which when combined with an appropriate substrate, will produce a detectable signal.

WM1158 cells are treated with increasing concentrations of test material for two hours. SDS-PAGE are conducted on cell lysates and Western Blot analysis are conducted on samples for total and phosphorylated AKT, MEK1/2, and ERK1/2

What is claimed is:

1. A pharmaceutical composition comprising a pharmaceutically acceptable excipient, carrier or diluent and a compound of Formula I: 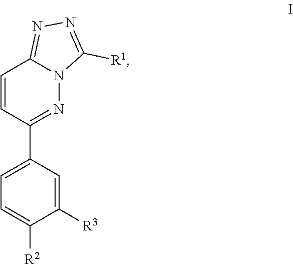
or a pharmaceutically acceptable salt thereof, wherein
R¹ is C₁₋₃ alkyl, phenyl, or 5-6 membered heteroaryl, wherein R¹ is optionally substituted by halo, —OH, C₁₋₄ alkyl, C₁₋₄ hydroxyalkyl, C₁₋₃ alkoxy, C₁₋₃ haloalkoxy, C₁₋₃ alkylcarbonyl, C₁₋₄ haloalkyl or C₁₋₃ haloalkylcarbonyl;
one of R² and R³ is hydrogen or fluoro, and the other of R² and R³ is —NH(CH₂)ₙCOR⁴ or —NH(CH₂)ₙSO₂R⁴;
n is an integer from zero to three;
R⁴ is phenyl or naphthyl optionally substituted by one, two or three R⁵, or R⁴ is 5-10 membered heteroaryl optionally substituted by one or two R⁵, and
each R⁵ is independently selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CON(C₁₋₄ alkyl)₂, —CO₂(C₁₋₄ alkyl), —NH(C₁₋₄ alkyl), —N(C₁₋₄ alkyl)₂, and C₁₋₄ haloalkoxy.

2. The pharmaceutical composition of claim 1, wherein:
R¹ is C₁₋₃ alkyl, phenyl, or 5-6 membered heteroaryl, wherein R¹ is optionally substituted by halo, —OH or C₁₋₃ alkoxy;
one of R² and R³ is hydrogen or fluoro, and the other of R² and R³ is —NH(CH₂)ₙCOR⁴ or —NH(CH₂)ₙSO₂R⁴;
n is an integer from zero to three;
R⁴ is phenyl or naphthyl optionally substituted by one, two or three R⁵, or R⁴ is 5-10 membered heteroaryl optionally substituted by one or two R⁵, and
each R⁵ is independently selected from halo, C₁₋₄ alkyl, C₁₋₄ haloalkyl, C₁₋₄ alkoxy and C₁₋₄ haloalkoxy. (claim 1 from first provisional to preserve priority).

3. The pharmaceutical composition of claim 1, wherein:
R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁-C₄ hydroxyalkyl, C₁₋₃ alkylcarbonyl or C₁₋₃ haloalkylcarbonyl;
R² is —NH(CH₂)ₙCOR⁴ or —NH(CH₂)ₙSO₂R⁴;
R³ is H;
R⁴ is phenyl, naphthyl, quinolinyl, pyridyl, furanyl, oxazolyl, thiazolyl, imidazolyl, thienyl or indolyl, each optionally substituted by one or two R⁵; and
each R⁵ is independently selected from F, Br, Cl, methyl, ethyl, isopropyl, S-methyl, S-ethyl, S-isopropyl, nitro, methoxy, ethoxy, propoxy and butoxy.

4. The pharmaceutical composition of claim 1, wherein:
R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁-C₄ hydroxyalkyl, C₁₋₃ alkylcarbonyl or C₁₋₃ haloalkylcarbonyl;
R² is —H;
R³ is NH(CH₂)ₙCOR⁴ or —NH(CH₂)ₙSO₂R⁴;
R⁴ is phenyl, naphthyl, quinolinyl, pyridyl, furanyl or indolyl, each optionally substituted by one or two R⁵; and
each R⁵ is independently selected from F, Br, Cl, methyl, ethyl, propyl, S-methyl, S-ethyl, S-isopropyl, nitro, methoxy, ethoxy, propyl and butoxy.

5. The pharmaceutical composition of claim 1 wherein:
R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁₋₄ hydroxyalkyl, C₁₋₃ alkylcarbonyl or C₁₋₃ haloalkylcarbonyl, e.g., C₁₋₄ alkyl or C₁-C₄ hydroxyalkyl;
R² is hydrogen;
R³ is —NHCOR⁴ or —NHSO₂R⁴; and
R⁴ is a phenyl or pyridyl, each optionally substituted with one or two R⁵.

6. The pharmaceutical composition of claim 1 wherein:
R¹ is methyl, phenyl, pyridyl, oxazolyl, thiazolyl, furanyl, pyrrolyl, pyrazolyl, or thienyl, e.g., methyl, pyridyl, thiazolyl, furanyl or thienyl, wherein the oxazolyl, thiazolyl, furanyl, pyrrolyl, pyrazolyl, and thienyl are optionally substituted with C₁₋₄ alkyl, C₁₋₄ haloalkyl, C₁₋₄ hydroxyalkyl, C₁₋₃ alkylcarbonyl or C₁₋₃ haloalkylcarbonyl, e.g., C₁₋₄ alkyl or C₁-C₄ hydroxyalkyl;
R² is —NHCOR⁴ or —NHSO₂R⁴;
R³ is hydrogen; and
R⁴ is an phenyl or pyridyl, each optionally substituted with one or two R⁵.

7. The pharmaceutical composition of claim 5 or 6 wherein:
R⁴ is phenyl substituted by one or two R⁵ independently selected from fluoro, chloro, bromo, —S(methyl), —S(ethyl), —S(isopropyl), methyl, ethyl, trifluoromethyl, methoxy and ethoxy, or R⁴ is a pyridyl optionally substituted by one R⁵ selected from fluoro, chloro, —S(isopropyl), methyl, methoxy or ethoxy.

8. The pharmaceutical composition of claim 1 wherein:
R¹ is pyridyl, 5-methyl-furan-2-yl, thiazol-5-yl, or 2-methylthiazol-5-yl, 2-methyl-oxazol-5-yl or oxazol-2-yl;
R² is hydrogen;
R³ is NHCOR⁴; and
R⁴ is 3-chlorophenyl, 3-SCH(CH₃)₂-phenyl or 3-ethyl-4-fluorophenyl.

9. The pharmaceutical composition of claim 1 wherein:
R¹ is 5-methyl-furan-2-yl;
one of R² or R³ is —H and the other is —NHSO₂R⁴ or —NHCOR⁴;
R⁴ is phenyl substituted with one or two R⁵; and
each R⁵ is independently selected from fluoro, chloro, methyl, ethyl, S-isopropyl, isobutyl, isopentyl, methoxy ethoxy.

10. The pharmaceutical composition of any one of claims 5, 6 or 8 wherein each R⁵ is independently selected from F, Br, Cl, —S(isopropyl), methyl, ethyl, propyl, isobutyl, nitro, methoxy, ethoxy, propoxy and butoxy.

11. The pharmaceutical composition of claim 1, wherein the compound is of Formula I-B-1: 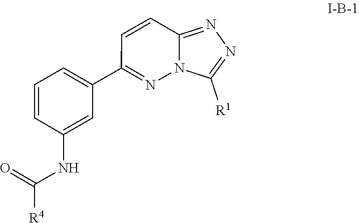
or a pharmaceutically acceptable salt thereof, wherein:
R¹ is a 5-6 membered heteroaryl ring optionally substituted by C₁₋₄ alkyl, C₁₋₄ hydroxyalkyl, C₁₋₃ alkoxy, C₁₋₃ haloalkoxy, C₁₋₃ alkylcarbonyl, haloalkyl or haloalkylcarbonyl;
R⁴ is phenyl or pyridyl substituted by one or two R⁵; and
each R⁵ is independently selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, or C₁₋₄ haloalkoxy.

12. The pharmaceutical composition of claim 11 wherein R¹ is pyridyl, furanyl, thiazolyl, pyrrolyl, pyrazolyl or oxazolyl, each optionally substituted with C₁₋₄ alkyl, C₁₋₄ hydroxyalkyl, C₁₋₃ alkoxy, C₁₋₃ haloalkoxy, C₁₋₃ alkylcarbonyl, haloalkyl or haloalkylcarbonyl (e.g., C₁₋₄ alkyl or C₁₋₄ hydroxyalkyl).

13. The pharmaceutical composition of claim 11 wherein R¹ is selected from:

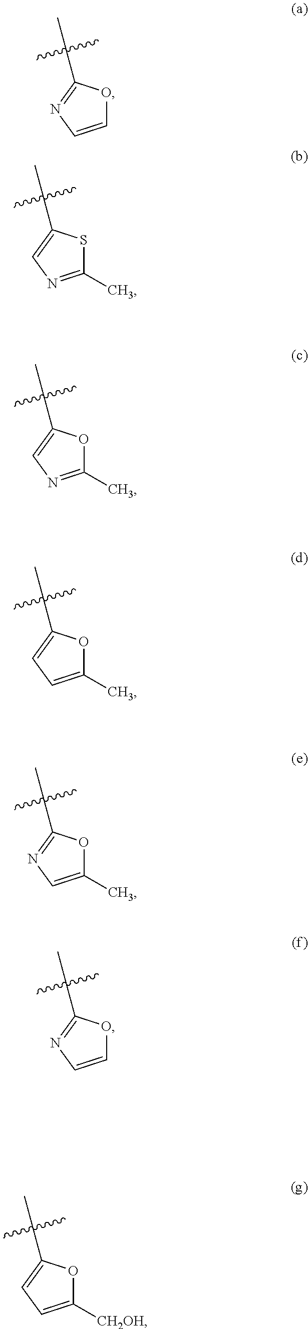

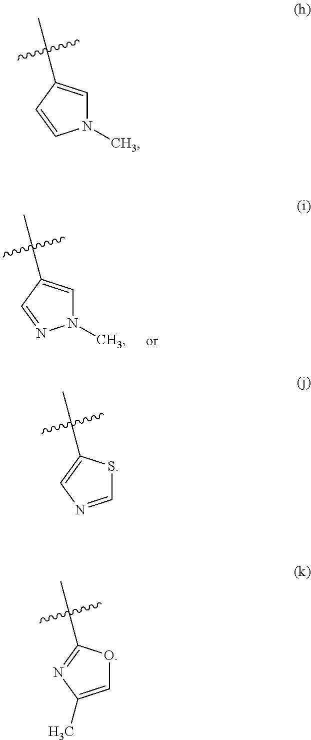

14. The pharmaceutical composition of claim 13 where there is a first R⁵ selected from halo, C₁₋₅ alkyl, —S(C₁₋₄ alkyl), C₁₋₅ haloalkyl, C₁₋₄ alkoxy, —SO(C₁₋₄ alkyl), —SO₂(C₁₋₄ alkyl), —CO(C₁₋₄ alkyl), —CONH(C₁₋₄ alkyl), —CO₂(C₁₋₄ alkyl), —NH(C₁₋₄ alkyl), —N(C₁₋₄ alkyl)₂, and C₁₋₄ haloalkoxy and optionally a second R⁵ selected from halo, C₁₋₅ alkyl, C₁₋₅ haloalkyl, and C₁₋₄ alkoxy.

15. The pharmaceutical composition of claim 14 where R⁴ is phenyl substituted in the meta-position by the first R⁵ and optionally substituted in the para-position by the second R⁵.

16. A method of treating cancer in a patient comprising administering to the patient an effective amount of the pharmaceutical composition of any one of claims 1-15.

17. The method of claim 16 wherein the cancer is characterized by overexpression of Rac1 or a genomic variant thereof.

18. The method of claim 17 wherein the Rac1 genomic variant is Rac1b or Rac1 P29S.

19. The method of any one of claims 16-18 wherein the cancer is selected from breast cancer, prostate cancer, ovarian cancer, melanoma, colorectal cancer, and renal cell carcinoma.

20. The method of claim 19 wherein the cancer is ER+ or HER2+ breast cancer.
